SEQUENCE LISTING (36 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3840 bp, SARS-CoV-2
ggtaccgccaccatgtttgtctttctggtcctgctgcccctggtgtcctcacagtgcgtcaatctgactactcgaactcagctgccccccgcttatactaattccttcacccggggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaattgcacatttgagtacgtgtcccagcctttcctgatggacctggagggcaagcagggcaatttcaagaacctgagggagttcgtgtttaagaatatcgatggctacttcaaaatctacagcaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgcaggagctgccgcctactatgtgggctatctgcagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaaccaggttcgccagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcacctggacagacaggcaagatcgccgactacaattataagctgccagacgatttcaccggctgcgtgatcgcctggaacagcaacaatctggattccaaggtcggcggcaactacaattatctgtaccggctgtttagaaagagcaatctgaagcccttcgagagggacatctctacagaaatctaccaggccggcagcaccccttgcaatggcgtggagggctttaactgttatttcccactgcagtcctacggcttccagcccacaaacggcgtgggctatcagccttaccgcgtggtggtgctgagctttgagctgctgcacgcaccagcaacagtgtgcggacccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacaccaggcaccaatacaagcaaccaggtggccgtgctgtatcaggacgtgaattgtaccgaggtgcctgtggccatccacgccgatcagctgaccccaacatggcgggtgtacagcaccggctccaacgtgttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagcccgggacctgatctgcgcccagaagtttaatggcctgaccgtgctgccacccctgctgacagatgagatgatcgcacagtacacaagcgccctgctggccggcaccatcacatccggatggaccttcggcgcaggagccgccctgcagatcccctttgccatgcagatggcctataggttcaacggcatcggcgtgacccagaatgtgctgtacgagaaccagaagctgatcgccaatcagtttaactccgccatcggcaagatccaggacagcctgtcctctacagcctccgccctgggcaagctgcaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcacaggcagactgcagtctctgcagacctatgtgacacagcagctgatcagggcagcagagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaataccttcgtgagcggcaactgtgacgtggtcatcggcatcgtgaacaataccgtgtatgatccactgcagcccgagctggacagctttaaggaggagctggataagtacttcaagaatcacacctcccctgacgtggatctgggcgacatcagcggcatcaatgcctccgtggtgaacatccagaaggagatcgaccgcctgaacgaggtggccaagaatctgaacgagagcctgatcgatctgcaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgatgaggacgactccgaacccgtgctgaagggcgtgaagctgcattacacctgactcgag

SEQ ID NO: 3 - DNA, 22 bp, Artificial Sequence
  synthetic primer
gttccagaactgcaccgaccct

SEQ ID NO: 4 - DNA, 22 bp, Artificial Sequence
  synthetic primer
agggtcggtgcagttctggaac

SEQ ID NO: 5 - DNA, 23 bp, Artificial Sequence
  synthetic primer
cgagttcaacgtgacctcttcag

SEQ ID NO: 6 - DNA, 23 bp, Artificial Sequence
  synthetic primer
ctgaagaggtcacgttgaactcg

SEQ ID NO: 7 - DNA, 22 bp, Artificial Sequence
  synthetic primer
catggaataggacgcgcatctc

SEQ ID NO: 8 - DNA, 22 bp, Artificial Sequence
  synthetic primer
gagatgcgcgtcctattccatg

SEQ ID NO: 9 - DNA, 24 bp, Artificial Sequence
  synthetic primer
ctgtacaactcaacctccttcagc

SEQ ID NO: 10 - DNA, 24 bp, Artificial Sequence
  synthetic primer
gctgaaggaggttgagttgtacag

SEQ ID NO: 11 - DNA, 21 bp, Artificial Sequence
  synthetic primer
ctagccgatggaccgcaggag

SEQ ID NO: 12 - DNA, 21 bp, Artificial Sequence
  synthetic primer
ctcctgcggtccatccgctag

SEQ ID NO: 13 - DNA, 22 bp, Artificial Sequence
  synthetic primer
gctgccagacaatttcaccgcc

SEQ ID NO: 14 - DNA, 22 bp, Artificial Sequence
  synthetic primer
gccggtgaaattgtctggcagc

SEQ ID NO: 15 - DNA, 27 bp, Artificial Sequence
  synthetic primer
gagctgctgaacgccacagctactgtg

SEQ ID NO: 16 - DNA, 27 bp, Artificial Sequence
  synthetic primer
cacagtagctgtggcgttcagcagctc

SEQ ID NO: 17 - DNA, 27 bp, Artificial Sequence
  synthetic primer
ctgactactcgaactcagctgcccccc

SEQ ID NO: 18 - DNA, 27 bp, Artificial Sequence
  synthetic primer
ggggggcagctgagttcgagtagtcag

SEQ ID NO: 19 - DNA, 27 bp, Artificial Sequence
  synthetic primer
cccgtgctgccttttaacgatggcgtg

SEQ ID NO: 20 - DNA, 27 bp, Artificial Sequence
  synthetic primer
cacgccatcgttaaaaggcagcacggg

SEQ ID NO: 21 - DNA, 21 bp, Artificial Sequence
  synthetic primer
tttaacgatggcgtgtacttc

SEQ ID NO: 22 - DNA, 21 bp, Artificial Sequence
  synthetic primer
gaagtacacgccatcgttaaa

SEQ ID NO: 23 - DNA, 22 bp, Artificial Sequence
  synthetic primer
gtactacaacaagaccaacaag

SEQ ID NO: 24 - DNA, 22 bp, Artificial Sequence
  synthetic primer
cttgttggtcttgttgtagtac

SEQ ID NO: 25 - DNA, 27 bp, Artificial Sequence
  synthetic primer
ctgatggacctggagggcaagcagggc

SEQ ID NO: 26 - DNA, 27 bp, Artificial Sequence
  synthetic primer
gccctgcttgccctccaggtccatcag

SEQ ID NO: 27 - DNA, 27 bp, Artificial Sequence
  synthetic primer
gagggcaagcagggcaatttcaagaac

SEQ ID NO: 28 - DNA, 27 bp, Artificial Sequence
  synthetic primer
gttcttgaaattgccctgcttgccctc

SEQ ID NO: 29 - DNA, 21 bp, Artificial Sequence
  synthetic primer
ggcaatttcaagaacctgagg

SEQ ID NO: 30 - DNA, 21 bp, Artificial Sequence
  synthetic primer
cctcaggttcttgaaattgcc

SEQ ID NO: 31 - DNA, 27 bp, Artificial Sequence
  synthetic primer
atcaacctggtgcgcgacctgcctcag

SEQ ID NO: 32 - DNA, 27 bp, Artificial Sequence
  synthetic primer
ctgaggcaggtcgcgcaccaggttgat

SEQ ID NO: 33 - DNA, 21 bp, Artificial Sequence
  synthetic primer
ctgcctcagggcttcagcgcc

SEQ ID NO: 34 - DNA, 21 bp, Artificial Sequence
  synthetic primer
ggcgctgaagccctgaggcag

SEQ ID NO: 35 - DNA, 21 bp, Artificial Sequence
  synthetic primer
acacccggcgactcctctagc

SEQ ID NO: 36 - DNA, 21 bp, Artificial Sequence
  synthetic primer
gctagaggagtcgccgggtgt

(19) United States
(12) Patent Application Publication
(10) Pub. No.: US 2024/0181042 A1
(43) Pub. Date: Jun. 6, 2024

(54) SARS-COV-2 VACCINE COMPOSITION AND USE THEREOF

(71) Applicants: National Tsing Hua University, Hsinchu (TW); Fu Jen Catholic University, New Taipei City (TW)

(72) Inventors: Suh-Chin Wu, Hsinchu (TW); I-Chen Chen, Hsinchu (TW); Wei-Shuo Lin, Hsinchu (TW); Yi-Chien Lee, New Taipei City (TW); Hao-Chan Hong, Hsinchu (TW)

(21) Appl. No.: 18/284,732

(22) PCT Filed: Apr. 1, 2022
(86) PCT No.: PCT/CN2022/084807

§ 371 Date: Sep. 28, 2023

Related U.S. Application Data
(63) Continuation of application No. 63/169,268, filed on Apr. 1, 2021.

(51) Int. Cl. A61K 39/215; A61K 39/00; A61P 31/14; C07K 14/005; C12N 7/00; C12N 15/86

(52) U.S. Cl. CPC A61K 39/215; A61P 31/14; C07K 14/005; C12N 7/00; C12N 15/86; A61K 2039/70; C12N 2710/10043; C12N 2770/20022; C12N 2770/20034

(57) ABSTRACT

The present disclosure provides a SARS-CoV-2 vaccine composition and use thereof. The SARS-CoV-2 vaccine composition includes a mutant SARS-CoV-2 spike protein with N-linked glycosylation in N-terminal domain or receptor binding domain, and can effectively elicit an immune response in an individual against different SARS-CoV-2 variants.

### CROSS-REFERENCE TO RELATED APPLICATION

This application claims priority of U.S. patent application No. 63/169,268, filed on Apr. 1, 2021, the content of which is incorporated herein in its entirety by reference.

### STATEMENT REGARDING SEQUENCE LISTING

[0001-1] The sequence listing associated with this application is provided in text format in lieu of a paper copy and is hereby incorporated by reference into the specification. The name of the TXT file containing the sequence listing is sl.txt. The TXT file is 22,770 bytes; was created on Oct. 2, 2023.

### BACKGROUND OF THE INVENTION

### 1. Field of the Invention

The present invention relates to a SARS-CoV-2 vaccine composition and use thereof, particularly relates to a SARS-CoV-2 vaccine composition using a mutant SARS-CoV-2 spike protein with N-linked glycosylation masking in the N-terminal domain or receptor binding domain as an antigen and use thereof.

### 2. The Prior Art

Severe acute respiratory syndrome coronavirus 2 (SARS-CoV-2) is an enveloped positive-stranded single-stranded RNA virus that belongs to the family Coronaviridae, genus Betacoronavirus, Severe acute respiratory syndrome-related coronavirus species. Virus particles are round or oval in shape, with a diameter of about 8054120 nanometers. Virus particles are wrapped by a double layer of phospholipids provided by the host cell, which mainly includes four structural proteins, including envelope protein (E protein), membrane protein (M protein), nucleocapsid (N protein), and spike protein (S protein). SARS-CoV-2 caused a severe infectious pneumonia (COVID-19) that broke out at the end of 2019. It can invade the human body through the human upper respiratory tract and use angiotensin-converting enzyme 2 (ACE2) expressed on the surface of various cells as the receptor to achieve infection. The main infected organs include the lungs, heart, kidneys and other major organs.

In order to avoid major health and economic losses caused by SARS-CoV-2, medical-related researchers focus on developing the SARS-CoV-2 vaccine. However, because SARS-CoV-2 is an RNA virus and has a high mutation rate, there are currently multiple SARS-CoV-2 variants spreading around the world, in which the British variant (Alpha, B.1.1.7), the South African variant (Beta, B.1.351) and the Indian variant (Delta, B.1.617.2) are the most serious.

In summary, it is indeed necessary to develop a vaccine composition that can broadly induce immune responses against different SARS-CoV-2 variants in order to cope with the infection and spread caused by the highly variable SARS-CoV-2.

### SUMMARY OF THE INVENTION

A primary objective of the present invention is to provide a mutant SARS-CoV-2 spike protein, comprising an N-linked glycosylation masking N-terminal domain and/or receptor-binding domain of SARS-CoV-2 spike protein.

According to an embodiment of the present invention, the aforementioned mutant SARS-CoV-2 spike protein has a mutation at an amino acid residue of a wild-type SARS-CoV-2 spike protein, wherein the amino acid residue is selected from the group consisting of: amino acid residue 21, amino acid residue 23, amino acid residue 85, amino acid residue 87, amino acid residue 89, amino acid residue 135, amino acid residue 137, amino acid residue 146, amino acid residue 148, amino acid residue 158, amino acid residue 160, amino acid residue 179, amino acid residue 181, amino acid residue 183, amino acid residue 185, amino acid residue 187, amino acid residue 213, amino acid residue 215, amino acid residue 219, amino acid residue 253, amino acid residue 354, amino acid residue 356, amino acid residue 370, amino acid residue 413, amino acid residue 428, amino acid residue 519, and amino acid residue 521. The mutation can be that substituting the amino acid residue with asparagine or threonine.

According to an embodiment of the present invention, the aforementioned amino acid residue 21 and the amino acid residue 23 of the wild-type SARS-CoV-2 spike protein have asparagine and threonine substitutions respectively, the amino acid residue 85 and the amino acid residue 87 have asparagine and threonine substitutions respectively, the amino acid residue 89 has threonine substitution, the amino acid residue 135 and the amino acid residue 137 have asparagine and threonine substitutions respectively, the amino acid residue 146 and the amino acid residue 148 have asparagine and threonine substitutions respectively, the amino acid residue 158 and the amino acid residue 160 have asparagine and threonine substitutions respectively, the amino acid residue 179 and the amino acid residue 181 have asparagine and threonine substitutions respectively, the amino acid residue 183 and the amino acid residue 185 have asparagine and threonine substitutions respectively, the amino acid residue 187 has threonine substitution, the amino acid residue 213 and the amino acid residue 215 have asparagine and threonine substitutions respectively, the amino acid residue 219 has asparagine substitution, the amino acid residue 253 has asparagine substitution, the amino acid residue 356 has threonine substitution, the amino acid residue 372 has threonine substitution, the amino acid residue 413 has asparagine substitution, the amino acid residue 428 has asparagine substitution, or the amino acid residue 519 and the amino acid residue 521 have asparagine and threonine substitutions respectively.

Another objective of the present invention is to provide a nucleic acid molecule, comprising a nucleotide sequence encoding the aforementioned mutant SARS-CoV-2 spike protein.

Another objective of the present invention is to provide a vaccine composition, comprising the aforementioned mutant SARS-CoV-2 spike protein.

According to an embodiment of the present invention, the mutant SARS-CoV-2 spike protein can be expressed on a recombinant virus, and the recombinant virus can comprise the aforementioned nucleic acid molecule.

According to an embodiment of the present invention, the recombinant virus can be a recombinant adenovirus.

Another objective of the present invention is to provide a use of the aforementioned mutant SARS-CoV-2 spike protein for preparing a SARS-CoV-2 vaccine composition.

According to an embodiment of the present invention, the SARS-CoV-2 vaccine composition can elicit an immune response against multiple SARS-CoV-2 variants in an individual.

According to an embodiment of the present invention, the SARS-CoV-2 vaccine composition can elicit high-titer antigen-specific antibodies and/or neutralizing antibodies.

In the SARS-CoV-2 vaccine composition of the present invention, a mutant SARS-CoV-2 spike protein that is overly glycosylated in the NTD or RBD is used to glycan mask unimportant epitopes, so that the response of individual B cells to the antibody of the SARS-CoV-2 spike protein can refocused without affecting the overall folding structure of the spike protein. The mutant SARS-CoV-2 spike protein of the present invention can effectively induce neutralizing antibody titers in individuals against the original Wuhan strain, the British variant, the South African variant, and the Indian variant of the SARS-CoV-2, to effectively improve an individual's ability to resist infection by different variants of SARS-CoV-2.

The embodiments of the present invention would be further described below with reference to the drawings. The examples listed below are used to illustrate the features and uses of the present invention, but are not intended to limit the scope of the present invention. Anyone skilled in the art can make some modifications and changes without departing from the spirit and scope of the invention. Therefore, the scope of the present invention shall be determined by the appended claims.

### BRIEF DESCRIPTION OF THE DRAWINGS

FIGS. 1A and 1B show a schematic diagram of the components of the SARS-CoV-2 spike protein. S represents spike protein; N′ represents N-terminal; C′ represents C-terminal; S1 represents S1 subunit; S2 represents S2 subunit; NTD represents N-terminal domain; RBD represents receptor-binding domain; S1/S2 represents furin cleavage site; FP represents fusion peptide; HR1 represents heptad repeat 1; HR2 represents heptad repeat 2; TM represents transmembrane domain; CT represents cytoplasmic tail; S2′ represents proteolytic cleavage site; The Y shape above represents original N-linked glycans; The Y-shape below represents additional added engineered glycan-masking, and #1 to #17 respectively represent different residues.

FIGS. 2A and 2B show schematic diagrams of the intact trimeric structure of the SARS-CoV-2 spike protein. NTD represents N-terminal domain; RBD represents receptor-binding domain; #1 represents the F135N/N137T residue, #2 represents the R158N/Y160T residue, #3 represents the N354/K356T residue, #4 represents the N370/A372T residue, #5 represents the G413N residue, #6 represents the D428N residue, #7 represents the H519N/P521T residue, #8 represents the R21N/Q23T residue, #9 represents the P85N/N87T residue, #10 represents the N87/G89T residue, #11 represents the H146N/N148T residue, #12 represents the L179N/G181T residue, #13 represents the Q183N/N185T residue, #14 represents the N185/K187T residue, #15 represents the V213N/D215T residue, #16 represents the G219N residue, and #17 represents that the D253N residue has additional glycan-masking sites.

FIGS. 3A and 3B show the results of detecting spike protein expressed in adenovirus vector using Western blotting. S represents spike protein; S1 represents S1 subunit.

FIG. 4A shows the anti-S IgG titer against the original Wuhan strain of SARS-CoV-2 in the serum of mice injected with the vaccine composition of the present invention. In FIGS. 4A to 4D, mice are immunized with the vaccine composition of the present invention containing Ad-S-F135N/N137T, Ad-S-R158N/Y160T, Ad-S-N370/A372T, or Ad-S-H519N/P521T.

FIG. 4B shows the anti-RBD IgG titer against the original Wuhan strain of SARS-CoV-2 in the serum of mice injected with the vaccine composition of the present invention.

FIG. 4C shows the pseudo-neutralization curves for the original Wuhan strain of SARS-CoV-2 in the serum of mice injected with the vaccine composition of the present invention.

FIG. 4D shows the IC50 neutralization titer (NT titer) of antibodies against the original Wuhan strain of SARS-CoV-2 in the serum of mice injected with the vaccine composition of the present invention.

FIG. 5A shows the anti-S IgG titer against the original Wuhan strain of SARS-CoV-2 in the serum of mice injected with the vaccine composition of the present invention. In FIGS. 5A to 5D, mice are immunized with the vaccine composition of the present invention containing Ad-S-N354/K356T, Ad-S-G413N, or Ad-S-D428N.

FIG. 5B shows the anti-RBD IgG titer against the original Wuhan strain of SARS-CoV-2 in the serum of mice injected with the vaccine composition of the present invention.

FIG. 5C shows the pseudo-neutralization curves for the original Wuhan strain of SARS-CoV-2 in the serum of mice injected with the vaccine composition of the present invention.

FIG. 5D shows the IC50 neutralization titer (NT titer) of antibodies against the original Wuhan strain of SARS-CoV-2 in the serum of mice injected with the vaccine composition of the present invention.

FIG. 6A shows the anti-S IgG titer against the original Wuhan strain of SARS-CoV-2 in the serum of mice injected with the vaccine composition of the present invention. In FIGS. 6A to 6D, mice are immunized with the vaccine composition of the present invention containing Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, or Ad-S-D253N.

FIG. 6B shows the anti-RBD IgG titer against the original Wuhan strain of SARS-CoV-2 in the serum of mice injected with the vaccine composition of the present invention.

FIG. 6C shows the pseudo-neutralization curves for the original Wuhan strain of SARS-CoV-2 in the serum of mice injected with the vaccine composition of the present invention.

FIG. 6D shows the IC50 neutralization titer (NT titer) of antibodies against the original Wuhan strain of SARS-CoV-2 in the serum of mice injected with the vaccine composition of the present invention.

FIG. 7A shows the anti-S IgG titer against the British variant of SARS-CoV-2 in the serum of mice injected with the vaccine composition of the present invention. In FIGS. 7A to 7D, mice are immunized with the vaccine composition of the present invention containing Ad-S-F135N/N137T, Ad-S-R158N/Y160T, Ad-S-N370/A372T, or Ad-S-H519N/P521T.

FIG. 7B shows the anti-RBD IgG titer against the British variant of SARS-CoV-2 in the serum of mice injected with the vaccine composition of the present invention.

FIG. 7C shows the pseudo-neutralization curves for the British variant of SARS-CoV-2 in the serum of mice injected with the vaccine composition of the present invention.

FIG. 7D shows the IC50 neutralization titer (NT titer) of antibodies against the British variant of SARS-CoV-2 in the serum of mice injected with the vaccine composition of the present invention.

FIG. 8A shows the anti-S1 IgG titer against the British variant of SARS-CoV-2 in the serum of mice injected with the vaccine composition of the present invention. In FIGS. 8A to 8D, mice are immunized with the vaccine composition of the present invention containing Ad-S-N354/K356T, Ad-S-G413N, or Ad-S-D428N.

FIG. 8B shows the anti-RBD IgG titer against the British variant of SARS-CoV-2 in the serum of mice injected with the vaccine composition of the present invention.

FIG. 8C shows the pseudo-neutralization curves for the British variant of SARS-CoV-2 in the serum of mice injected with the vaccine composition of the present invention.

FIG. 8D shows the IC50 neutralization titer (NT titer) of antibodies against the British variant of SARS-CoV-2 in the serum of mice injected with the vaccine composition of the present invention.

FIG. 9A shows the pseudo-neutralization curves for the British variant of SARS-CoV-2 in the serum of mice injected with the vaccine composition of the present invention. In FIGS. 9A and 9B, mice are immunized with the vaccine composition of the present invention containing Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, or Ad-S-D253N.

FIG. 9B shows the IC50 neutralization titer (NT titer) of antibodies against the British variant of SARS-CoV-2 in the serum of mice injected with the vaccine composition of the present invention.

FIG. 10A shows the anti-S1 IgG titer against the South African variant of SARS-CoV-2 in the serum of mice injected with the vaccine composition of the present invention. In FIGS. 10A to 10D, mice are immunized with the vaccine composition of the present invention containing Ad-S-F135N/N137T, Ad-S-R158N/Y160T, Ad-S-N370/A372T, or Ad-S-H519N/P521T.

FIG. 10B shows the anti-RBD IgG titer against the South African variant of SARS-CoV-2 in the serum of mice injected with the vaccine composition of the present invention.

FIG. 10C shows the pseudo-neutralization curves for the South African variant of SARS-CoV-2 in the serum of mice injected with the vaccine composition of the present invention.

FIG. 10D shows the IC50 neutralization titer (NT titer) of antibodies against the South African variant of SARS-CoV-2 in the serum of mice injected with the vaccine composition of the present invention.

FIG. 11A shows the anti-S1 IgG titer against the South African variant of SARS-CoV-2 in the serum of mice injected with the vaccine composition of the present invention. In FIGS. 11A to 11D, mice are immunized with the vaccine composition of the present invention containing Ad-S-N354/K356T, Ad-S-G413N, or Ad-S-D428N.

FIG. 11B shows the anti-RBD IgG titer against the South African variant of SARS-CoV-2 in the serum of mice injected with the vaccine composition of the present invention.

FIG. 11C shows the pseudo-neutralization curves for the South African variant of SARS-CoV-2 in the serum of mice injected with the vaccine composition of the present invention.

FIG. 11D shows the IC50 neutralization titer (NT titer) of antibodies against the South African variant of SARS-CoV-2 in the serum of mice injected with the vaccine composition of the present invention.

FIG. 12A shows the pseudo-neutralization curves for the South African variant of SARS-CoV-2 in the serum of mice injected with the vaccine composition of the present invention. In FIGS. 12A and 12B, mice are immunized with the vaccine composition of the present invention containing Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, or Ad-S-D253N.

FIG. 12B shows the IC50 neutralization titer (NT titer) of antibodies against the South African variant of SARS-CoV-2 in the serum of mice injected with the vaccine composition of the present invention.

FIG. 13A shows the anti-S1 IgG titer against the Indian variant of SARS-CoV-2 in the serum of mice injected with the vaccine composition of the present invention. In FIGS. 13A to 13D, mice are immunized with the vaccine composition of the present invention containing Ad-S-F135N/N137T, Ad-S-R158N/Y160T, Ad-S-N370/A372T, or Ad-S-H519N/P521T.

FIG. 13B shows the anti-RBD IgG titer against the Indian variant of SARS-CoV-2 in the serum of mice injected with the vaccine composition of the present invention.

FIG. 13C shows the pseudo-neutralization curves for the Indian variant of SARS-CoV-2 in the serum of mice injected with the vaccine composition of the present invention.

FIG. 13D shows the IC50 neutralization titer (NT titer) of antibodies against the Indian variant of SARS-CoV-2 in the serum of mice injected with the vaccine composition of the present invention.

FIG. 14A shows the anti-S1 IgG titer against the Indian variant of SARS-CoV-2 in the serum of mice injected with the vaccine composition of the present invention. In FIGS. 14A to 14D, mice are immunized with the vaccine composition of the present invention containing Ad-S-N354/K356T, Ad-S-G413N, or Ad-S-D428N.

FIG. 14B shows the anti-RBD IgG titer against the Indian variant of SARS-CoV-2 in the serum of mice injected with the vaccine composition of the present invention.

FIG. 14C shows the pseudo-neutralization curves for the Indian variant of SARS-CoV-2 in the serum of mice injected with the vaccine composition of the present invention.

FIG. 14D shows the IC50 neutralization titer (NT titer) of antibodies against the Indian variant of SARS-CoV-2 in the serum of mice injected with the vaccine composition of the present invention.

FIG. 15A shows the pseudo-neutralization curves for the Indian variant of SARS-CoV-2 in the serum of mice injected with the vaccine composition of the present invention. In FIGS. 15A and 15B, mice are immunized with the vaccine composition of the present invention containing Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, or Ad-S-D253N.

FIG. 15B shows the IC50 neutralization titer (NT titer) of antibodies against the Indian variant of SARS-CoV-2 in the serum of mice injected with the vaccine composition of the present invention.

FIGS. 16A and 16B show a comparison of the neutralizing antibody titers of the vaccine composition of the present invention with different glycan-masking spike proteins against different SARS-CoV-2 variants after immunization. In FIG. 16A, mice are immunized with the vaccine composition of the present invention containing Ad-S-F135N/N137T, Ad-S-R158N/Y160T, Ad-S-N370/A372T, or Ad-S-H519N/P521T. In FIG. 16B, mice are immunized with the vaccine composition of the present invention containing Ad-S-N354/K356T, Ad-S-G413N, or Ad-S-D428N.

### DETAILED DESCRIPTION OF THE PREFERRED EMBODIMENT

### Definition

As used herein, the data provided represent experimental values that can vary within a range of 20%, preferably within 10%, and most preferably within 5%.

As used herein, when analyzing the data to detect the titers of anti-spike protein IgG antibodies and anti-RBD IgG antibodies, statistical tests for multiple comparison were performed for all groups (except for the PBS control) in case of the ELISA data. The results were analyzed using the nonparametric Kruskal-Wallis test, with corrected Dunn's multiple comparison test, using GraphPad Prism v6.01. Statistical significance has been expressed as follows: *p<0.05; **p<0.01; and ***p<0.001. When analyzing experimental data for neutralizing antibody titers, neutralization curves were fitted based on the equation of nonlinear regression log (inhibitor) vs. normalized response—variable slope. The IC-50 values of the neutralization were obtained from the fitting curves. All experiments were performed at least three times, and data are expressed as mean±standard deviation.

As used herein, the term “N-linked glycosylation” refers to the sugar chain of aspartic acid covalently connected to a protein with an N-glycosidic bond, including about at least ten different types of monosaccharide units. More specifically, the sugar chain is linked to asparagine (N) in an amino acid residue, and the amino acid residue is asparagine (N)-any amino acid (X)-Serine (S) or threonine (T), represented by N-X-S/T. N-linked glycosylation has different molecular weights and structures depending on the composition of monosaccharides.

As used herein, unless otherwise specified, the term “overglycated” means having additional “glycan-masking mutations” on amino acid residues in addition to the “native glycan-masking” amino acid residues on the wild-type protein.

As used herein, unless otherwise specified, the term “mutant” is equivalent to the term “variant”.

As used herein, R21N/Q23T, P85N/N87T, N87/G89T, F135N/N137T, H146N/N148T, R158N/Y160T, L179N/G181T, Q183N/N185T, N185/K187T, V213N/D215T, G219N, D253 N, N354/K356T, N370/A372T, G413N, D428N, and H519N/P521T represent the substitution of specific amino acid residues of the wild-type SARS-CoV-2 spike protein with asparagine and/or threonine to show the mutant SARS-CoV-2 spike protein of the present invention.

As used herein, Ad-S-F135N/N137T, Ad-S-R158N/Y160T, Ad-S-N354/K356T, Ad-S-N370/A372T, Ad-S-G413N, Ad-S-D428N, Ad-S-H519N/P521T, Ad-S-R21N/Q23T, Ad-S-P85N/N87, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, and Ad-S-D253N represent recombinant adenoviruses expressing different mutant SARS-CoV-2 spike proteins of the present invention.

According to the present invention, the operating procedures and parameter conditions related to gene cloning fall within the professionalism and routine technical scope of those skilled in the art.

According to the present invention, the operating procedures and parameter conditions related to site-directed mutagenesis fall within the professionalism and routine technical scope of those skilled in the art.

According to the present invention, the operating procedures and parameter conditions related to addition of N-linked glycosylation to amino acid residues of proteins fall within the professionalism and routine technical scope of those skilled in the art.

According to the present invention, the operating procedures and parameter conditions related to expression of antigens with adenovirus fall within the professionalism and routine technical scope of those skilled in the art, and as used herein, “adenovirus vector” refers to a recombinant adenovirus that expresses different mutant SARS-CoV-2 spike proteins of the present invention.

### Materials and Methods

### Experimental Cells and Culture Method

In embodiments of the present invention, human embryonic kidney cell line 293A (HEK293A) and human embryonic kidney cell line 293T (HEK293T) were used to perform cell experiments. HEK293A and HEK293T cells were obtained from the Bioresource Collection and Research Center (BCRC), Taiwan. These cells were grown in Dulbecco's modified Eagle medium (DMEM) (Thermo Scientific) supplemented with 10% fetal bovine serum (FBS) (Gibco) and 100 units/ml penicillin/streptomycin (P/S), and maintained in an incubator at 37° C. with 5% CO₂.

### Preparation of Adenovirus Vector

In embodiments of the present invention, adenovirus expressing the wild-type SARS-CoV-2 spike protein or the mutant spike protein with glycan-masking mutations was used as a vector to immunize experimental animals. Genes encoding wild-type spike protein (S) or mutant spike protein were first cloned into the pENTR1A vector (Invitrogen), and then cloned into the adenoviral plasmid pAd/CMV/V5-DEST (Invitrogen) using LR Clonase™ II Enzyme Mix (Invitrogen) to produce the adenoviral plasmid expressing wild-type spike protein (S) or mutant spike protein.

To obtain adenovirus vector expressing wild-type spike protein (S) or mutant spike protein, the adenoviral plasmids were cleaved with Pac I restriction enzyme to expose the inverted terminal repeats and then transfected into 293A cells separately using TurboFect transfection reagent (Fermentas). After 10-15 days, once the cytopathic effects (CPEs) were visible, the transfected cells and culture media were collected. The cells were disrupted by means of three freeze-thaw cycles to release the intracellular viral particles, and the supernatants of the cell lysates were collected by centrifugation (3000 rpm, 15 min, 4° C.) to obtain the adenovirus vectors expressing the SARS-Co-V-2 spike proteins. To prepare adenovirus vectors having higher titers, the virus was concentrated using a 30-kDa Amicon Ultra-15 Centrifugal Filter (Millipore). The viral stocks of the adenovirus vector were stored at −80° C.

To determine the titers of the adenovirus vector, HEK293A cells were seeded into 6-well plates at a density of 10⁶ cells/well and incubated at 37° C. overnight. The 10-fold serially diluted stocks of the adenovirus vector were then added to each well at 37° C. for 24 h. Next, the media containing the diluted adenovirus vectors were removed, and 3 mL/well of DMEM containing 0.4% agarose and 100 U/ml penicillin/streptomycin (P/S) was added to the 6-well plates. The plaques were visibly quantified 7-10 days after the cells were infected with adenovirus vectors, and the plaque-forming unit (PFU) count was noted.

### Operation of SDS-PAGE

The operation of sodium dodecyl sulfate polyacrylamide gel electrophoresis (SDS-PAGE) is briefly described as follows. First, the protein sample was mixed with reducing sample buffer (comprising 50 mM Tris-HCl, pH 6.8; 100 mM dithiothreonate (DTT); 2% SDS; 0.1% bromophenol blue; and 10% glycerol) at a ratio of 3:1 and heated at 95° C. for 5 minutes.

At the same time, electrophoretic gels including separating gel and stacking gel were prepared. Taking 12% separating gel as an example, it comprises 2.5 mL of 1 M Tris, pH 8.8; 3.3 mL of deionized water; 4 mL of 30% acrylamide mix; 0.1 mL of 10% SDS; 0.1 ml of 10% ammonium persulfate (APS); and 0.01 ml of tetramethylethylenediamine (TEMED). Taking 5% stacking gel as an example, it comprises 0.63 mL of 1 M Tris, pH 6.8; 3.4 mL of deionized water; 0.83 mL of 30% acrylamide premix; 0.05 mL of 10% SDS; 0.05 mL of 10% APS; and 0.005 mL of TEMED.

Protein electrophoresis was stacked at a voltage of 80V and separated at 140V. The time of electrophoresis depends on the molecular weight of the protein to be detected. The gel was stained with Coomassie brilliant blue dye solution (comprising 0.1% coomassie R250; 10% acetic acid; and 50% methanol) for 1 hour, and then destained with a destaining solution (comprising 10% acetic acid and 50% methanol).

Operation of Western blot

The operation of Western blot is briefly described as follows. In the transfer tank, the gel of the protein sample separated by SDS-PAGE was transferred to the nitrocellulose membrane (hereinafter referred to as NC membrane) at a voltage of 135V, and then the NC membrane containing the transferred protein was soaked in 20 mL of blocking solution and shaken for at least 1 hour to block non-specific binding. The blocking solution is Tris-buffered saline containing Tween-20 (hereinafter referred to as TBST solution) added with 5% skim milk, comprising 50 mM Tris; 150 mM sodium chloride; and 0.05% Tween-20.

After washing the NC membrane 3 times with TBST solution, primary antibody diluted with specific multiples in TBST solution was added, and shaken at 4° C. for about 16 hours. The next day, after washing with TBST solution three times, secondary antibodies conjugated with horseradish peroxidase (HRP) diluted at specific times in TBST solution were used, shaken at room temperature for 1 hour, followed by washing 3 times with TBST solution. An enhanced chemical luminescence reagent (HRP-catalyzed enhanced chemiluminescence, Millipore) was added to the film for 1 minute to generate a luminescence signal, and developed onto an X-ray film, such as medical X-ray film (Fujifilm).

### Immunization Methods for Experimental Mice

In one embodiment of the present invention, BALB/c female mice aged 6 to 8 weeks were used to perform vaccination experiments. Groups of female BALB/c mice were obtained from the National Laboratory Animal Center, Taipei, Taiwan. Groups of female BALB/c mice were immunized with Ad-S, Ad-S-F135N/N137T, Ad-S-R158N/Y160T, Ad-S-N370/A372T, or Ad-S-H519N/P521T vectors at 5×10⁷ plaque-forming unit (pfu) per dose in PBS (pH 7.4) in the first set of immunization experiments, and immunized with Ad-S, Ad-S-N354/K356T, Ad-S-G413N, and Ad-S-D428N vectors at 1×10⁸ pfu per dose in the second set of immunization experiments. Intramuscular injections were administered at weeks 0 and 3. Sera were collected 2 weeks after the second immunization dose.

Collection of Serum Samples from Experimental Mice

Mice were immunized with the aforementioned method, and serum samples of each mouse were collected 2 weeks after the second immunization injection. Before sampling, the mice were heated for 10 minutes under an ultra-red light and a heating blanket, and disinfected with 70% ethanol. Then, a scalpel was used to cut the lateral tail vein of the mouse and approximately 500 μL of blood was collected. The whole blood was stood at room temperature for 2 hours. After the blood was clotted, centrifugation was performed twice at 800 g for 15 minutes to remove blood clots, and the serum was immediately transferred to a new centrifuge tube. Heat treatment was performed at 56° C. for 30 minutes to inactivate complement. After cooling to room temperature, serum was dispensed and stored at −20° C.

### Preparation of SARS-CoV-2 Pseudotyped Lentivirus

The preparation method of the SARS-CoV-2 pseudotyped lentivirus is briefly as follows. Plasmid pcDNA3.1-nCoV-Ad18 expressing the full-length SARS-CoV-2 spike protein (Wuhan-Hu-1, B.1.1.7, or B.1.351), packaging and reporter plasmids pLAS2w.FLuc.Ppuro and pCMVAR8.91 (RNAi Core, Academia Sinica) were co-transfected into HEK293T cells using TransIT-LT1 transfection reagent (Mirus Bio). The culture medium was harvested and concentrated 48 hours after transfection. The titer of the pseudotyped lentivirus can be evaluated by detecting the luciferase activity transcribed in HEK293 cells that stably express ACE2 infected with the SARS-CoV-2 pseudotyped lentivirus.

### Example 1

### Preparation of Mutant SARS-CoV-2 Spike Protein of Present Invention

In one embodiment of the present invention, based on the three-dimensional structure of the SARS-CoV-2 spike protein, target sites suitable for additional glycan-masking were selected to mask unimportant epitopes, so that the response of B cells to the antibody of the spike protein can refocused without affecting the overall folding structure of the spike protein. An adenovirus vector was used to express the spike protein antigen with glycan-masking mutations at the target site as the main component of the vaccine composition of the present invention.

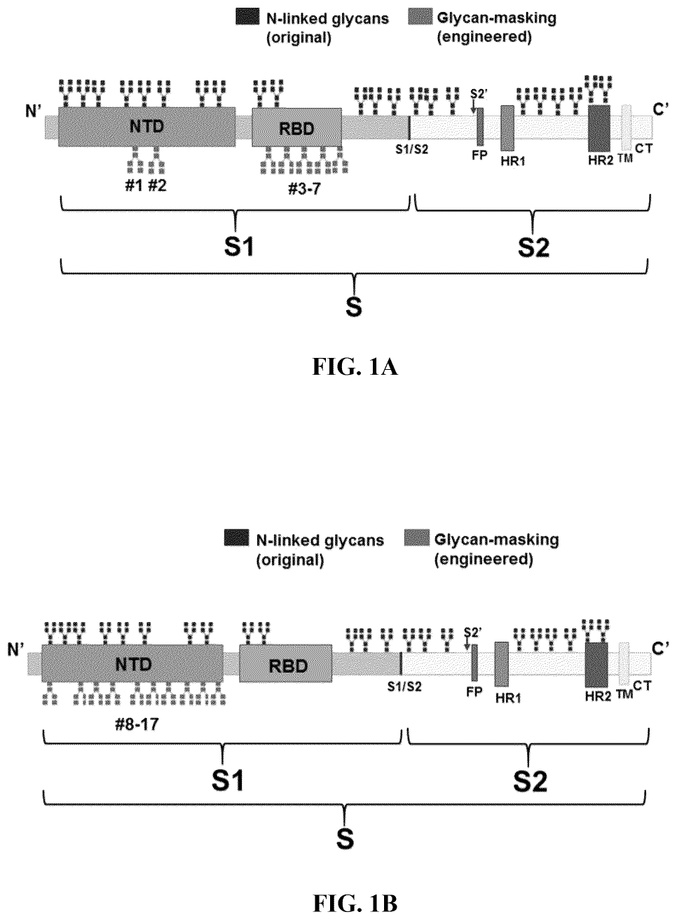

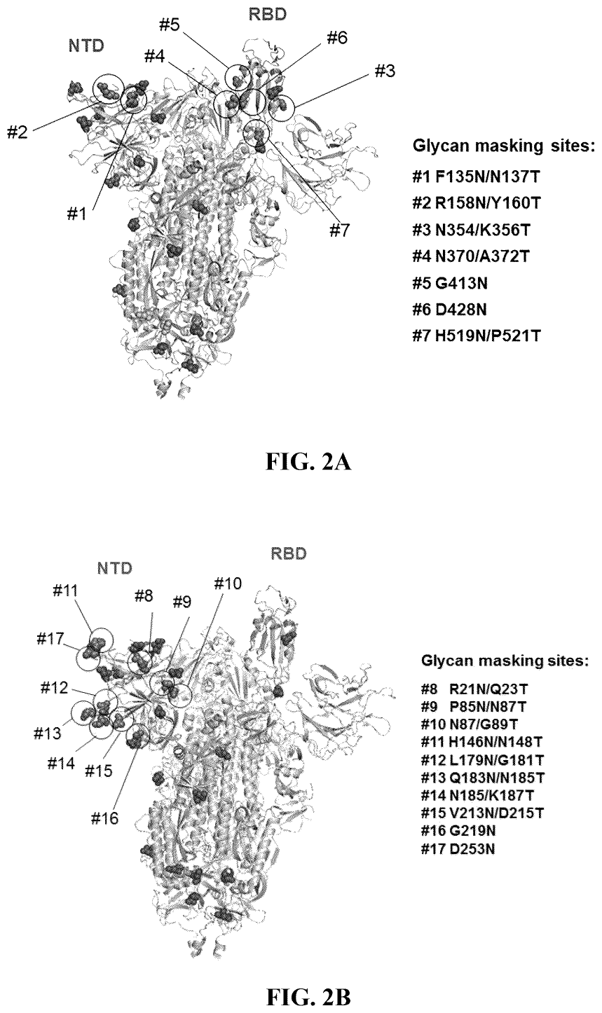

As shown in FIGS. 1A, 1B, 2A, and 2B, the SARS-CoV-2 spike protein is a trimer, and each monomer is composed of S1 subunits and S2 subunits. The S1 subunit comprises the N-terminal domain (NTD) and the receptor-binding domain (RBD). The main function of RBD is to bind to ACE2 on the surface of host cells, allowing SARS-CoV-2 to enter the host cells. Since RBD and NTD interact with each other in the quaternary structure of the intact trimeric spike protein, in the embodiment of the present invention, in addition to using RBD as the target of glycan-masking modification, glycan-masking sites located in NTD are also selected.

In the embodiment of the present invention, PyMol (The PyMol Molecular Graphics System, version 4.0; Schradinger, LLC) was used to confirm that in the three-dimensional structure of spike protein (PDB ID: 7C2L), the exposed loops or the protruding sites of the exposed loops on the NTD and RBD were used as target sites for glycan-masking addition. Sites with native glycan masking and RBD distances less than 5 Å were discarded. Finally, 17 groups of amino acid residues were screened and additional glycan masking modifications were added to prepare 17 mutant SARS-CoV-2 spike proteins of the present invention. Their N-linked glycosylation sites are shown in FIGS. 1A, 1B, 2A and 2B.

Compared to the amino acid sequence of the wild-type SARS-CoV-2 spike protein (SEQ ID NO: 1), the 17 mutant spike proteins have one or two amino acid substitutions to achieve N-linked glycosylation. Specifically, the amino acid sequence is made to appear in the order of asparagine-any amino acid-serine (S) or threonine (N-X-S/T), as shown in Table 1. Phenylalanine (F) on the amino acid residue 135 and asparagine on the amino acid residue 137 have asparagine and threonine substitutions respectively (#1 F135N/N137T); Arginine (R) on the amino acid residue 158 and tyrosine (Y) on the amino acid residue 160 have asparagine and threonine substitutions respectively (#2 R158N/Y160T); Lysine (K) on the amino acid residue 356 has threonine substitution (#3 N354/K356T); Alanine (A) on the amino acid residue 372 has threonine substitution (#4 N370/A372T); Glycine (G) on the amino acid residue 413 has asparagine substitution (#5 G413N); Aspartic acid (D) on the amino acid residue 428 has asparagine substitution (#6 D428N); Histidine (H) on the amino acid residue 519 and proline (P) on the amino acid residue 521 have asparagine and threonine substitutions respectively (#7 H519N/P521T); Arginine on the amino acid residue 21 and glutamine (Q) on the amino acid residue 23 have asparagine and threonine substitutions respectively (#8 R21N/Q23T); Proline on the amino acid residue 85 and asparagine on the amino acid residue 87 have asparagine and threonine substitutions respectively (#9 P85N/N87T); Glycine on the amino acid residue 89 has threonine substitution (#10 N87/G89T); Histidine on the amino acid residue 146 and asparagine on the amino acid residue 148 have asparagine and threonine substitutions respectively (#11 H146N/N148T); Leucine (L) on the amino acid residue 179 and glycine on the amino acid residue 181 have asparagine and threonine substitutions respectively (#12 L179N/G181T); Glutamine on the amino acid residue 183 and asparagine on the amino acid residue 185 have asparagine and threonine substitutions respectively (#13 Q183N/N185T); Lysine on the amino acid residue 187 has threonine substitution (#14 N185/K187T); Valine (V) on the amino acid residue 213 and aspartic acid on the amino acid residue 215 have asparagine and threonine substitutions respectively (#15 V213N/D215T); glycine on the amino acid residue 219 has asparagine substitution (#16 G219N); and aspartic acid on the amino acid residue 253 has asparagine substitution (#17 D253N).

TABLE 1

| | | Amino acid sequence of N-linked |
|---|---|---|
| No. | Mutant spike protein | glycosylated residues |
| #1 | F135N/N137T | ¹³⁵NCT¹³⁷ |
| #2 | R158N/Y160T | ¹⁵⁸NVT¹⁶⁰ |
| #3 | N354/K356T | ³⁵⁴NRT³⁵⁶ |
| #4 | N370/A372T | ³⁷⁰NST³⁷² |
| #5 | G413N | ⁴¹³NQT⁴¹⁵ |
| #6 | D428N | ⁴²⁸NFT⁴³⁰ |
| #7 | H519N/P521T | ⁵¹⁹NAT⁵²¹ |
| #8 | R21N/Q23T | ²¹NTT²³ |
| #9 | P85N/N87T | ⁸⁵NFT⁸⁷ |
| #10 | N87/G89T | ⁸⁷NDT⁸⁹ |
| #11 | H146N/N148T | ¹⁴⁶NKT¹⁴⁸ |
| #12 | L179N/G181T | ¹⁷⁹NET¹⁸¹ |
| #13 | Q183N/N185T | ¹⁸³NGT¹⁸⁵ |
| #14 | N185/K187T | ¹⁸⁵NFT¹⁸⁷ |
| #15 | V213N/D215T | ²¹³NRT²¹⁵ |
| #16 | G219N | ²¹⁹NFS²²¹ |
| #17 | D253N | ²⁵³NSS²⁵⁵ |

To construct an adenovirus expression vector comprising these genes encoding mutant spike proteins, the SARS-CoV-2 spike protein gene from GenScript (Wuhan-Hu-1 strain, accession number MN908947.3) was human codon-optimized (SEQ ID NO: 2). The primers (SEQ ID NO: 3 to SEQ ID NO: 36) shown in Table 2 below were used to perform site-directed mutagenesis based on polymerase chain reaction (PCR), to obtain DNA fragments comprising 17 mutant spike protein genes. Adenovirus vectors expressing the mutant spike proteins were prepared using the aforementioned preparation method of the adenovirus vector. They are marked as Ad-S-F135N/N137T, Ad-S-R158N/Y160T, Ad-S-N370/A372T, Ad-S-H519N/P521T, Ad-S-N354/K356T, Ad-S-G413N, Ad-S-D428N, Ad-S-H519N/P521T, Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, and Ad-S-D253N. At the same time, an adenovirus vector expressing the wild-type SARS-Co-V-2 spike protein was prepared as a comparison group, marked as Ad-S.

TABLE 2

| | Mutant spike | | SEQ ID |
|---|---|---|---|
| No. | protein | PCR primer sequence | NO. |
| #1 | F135N/N137T | F: gttccagaactgcaccgaccct | 3 |
| | | R: agggtcggtgcagttctggaac | 4 |
| #2 | R158N/Y160T | F: cgagttcaacgtgacctcttcag | 5 |
| | | R: ctgaagaggtcacgttgaactcg | 6 |
| #3 | N354/K356T | F: catggaataggacgcgcatctc | 7 |
| | | R: gagatgcgcgtcctattccatg | 8 |
| #4 | N370/A372T | F: ctgtacaactcaacctccttcagc | 9 |
| | | R: gctgaaggaggttgagttgtacag | 10 |
| #5 | G413N | F: ctagccgatggaccgcaggag | 11 |
| | | R: ctcctgcggtccatccgctag | 12 |
| #6 | D428N | F: gctgccagacaatttcaccgcc | 13 |
| | | R: gccggtgaaattgtctggcagc | 14 |
| #7 | H519N/P521T | F: gagctgctgaacgccacagctactgtg | 15 |
| | | R: cacagtagctgtggcgttcagcagctc | 16 |
| #8 | R21N/Q23T | F: ctgactactcgaactcagctgcccccc | 17 |
| | | R: ggggggcagctgagttcgagtagtcag | 18 |
| #9 | P85N/N87T | F: cccgtgctgccttttaacgatggcgtg | 19 |
| | | R: cacgccatcgttaaaaggcagcacggg | 20 |
| #10 | N87/G89T | F: tttaacgatggcgtgtacttc | 21 |
| | | R: gaagtacacgccatcgttaaa | 22 |
| #11 | H146N/N148T | F: gtactacaacaagaccaacaag | 23 |
| | | R: cttgttggtcttgttgtagtac | 24 |
| #12 | L179N/G181T | F: ctgatggacctggagggcaagcagggc | 25 |
| | | R: gccctgcttgccctccaggtccatcag | 26 |
| #13 | Q183N/N185T | F: gagggcaagcagggcaatttcaagaac | 27 |
| | | R: gttcttgaaattgccctgcttgccctc | 28 |
| #14 | N185/K187T | F: ggcaatttcaagaacctgagg | 29 |
| | | R: cctcaggttcttgaaattgcc | 30 |
| #15 | V213N/D215T | F: atcaacctggtgcgcgacctgcctcag | 31 |
| | | R: ctgaggcaggtcgcgcaccaggttgat | 32 |
| #16 | G219N | F: ctgcctcagggcttcagcgcc | 33 |
| | | R: ggcgctgaagccctgaggcag | 34 |
| #17 | D253N | F: acacccggcgactcctctagc | 35 |
| | | R: gctagaggagtcgccgggtgt | 36 |

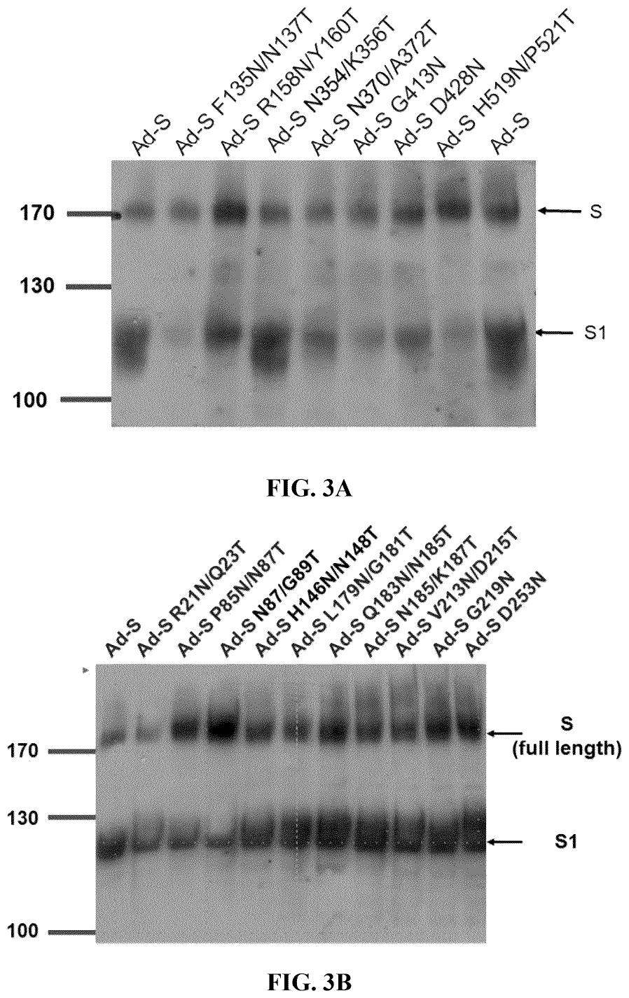

In order to confirm the spike protein expressed in the adenovirus vector, SDS-PAGE and Western blot were used to analyze whether there was spike protein and its S1 subunit in the cell lysate infected by the adenovirus vector. HEK293A cells were infected with Ad-S (adenovirus vector expressing the wild-type spike protein), Ad-S-F135N/N137T, Ad-S-R158N/Y160T, Ad-S-N370/A372T, Ad-S-H519N/P521T, Ad-N354/K356T, Ad-S-G413N, Ad-S-D428N, Ad-S-H519N/P521T, Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, and Ad-S-D253N at an MOI=5 for 48 h, then lysed with Glo Lysis buffer (Promega), and subjected to centrifugation at 12000×g for 5 min at 4° C. to remove the cell debris. Cell lysates were mixed with reducing sample buffer and heated at 95° C. for 5 minutes and allowed to be treated with PNGase F (BioLabs) at 37° C. for 2 hours. It can also be processed without PNGase F. The proteins in the sample were then separated by SDS-PAGE using a 7% or 8% separating gel. After the gel of SDS-PAGE were transferred to NC membrane (Millipore), blocking solution was used for 1 hour at room temperature, and then washed three times with TBST solution. The primary antibody anti-SARS-CoV-2 antibody (GTX135356, GeneTex) was added and the reaction was performed overnight, and the secondary antibody HRP-conjugated goat anti-rabbit IgG (KPL) was added and detected for 1 hour at room temperature. Chemical luminescence reagents were used to detect antibody signals and captured using an X-ray film. The results are shown in FIGS. 3A and 3B.

As can be seen from FIGS. 3A and 3B, after infection with Ad-S, Ad-S-F135N/N137T, Ad-S-R158N/Y160T, Ad-S-N370/A372T, Ad-S-H519N/P521T, Ad-S-N354/K356T, Ad-S-G413N, Ad-S-D428N, Ad-S-H519N/P521T, Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, and Ad-S-D253N in HEK293A cells, the SARS-CoV-2 spike protein and its S1 subunit do exist.

### Example 2

### Mutant SARS-CoV-2 Spike Protein of Present Invention Improves Titer of Antibodies Against Original Wuhan Strain

In one embodiment of the present invention, in order to confirm that the mutant SARS-CoV-2 spike protein of the present invention can effectively induce mammals to produce an antibody response against SARS-CoV-2, the vaccine composition was prepared using the adenovirus vector expressing the glycan-masking spike protein of the present invention, and injected into experimental mice. Adenovirus vector expressing wild-type spike protein was used as a comparison group. After a period of time, the serum of the mice was collected to analyze the antibody titer against the original Wuhan strain of SARS-CoV-2 (Wuhan-Hu-1, Ancestral).

First, adenovirus vectors expressing wild-type spike protein or mutant spike protein were diluted using PBS solution to prepare 100 μL of vaccine composition. BALB/c mice (n=5) were divided into the following groups for immune injection. (1) Control group (PBS): mice were only injected intramuscularly with PBS solution; (2) Comparison group (Ad-S): mice were intramuscularly injected with a vaccine composition comprising 5×10⁷ pfu of adenovirus vector expressing wild-type spike protein; (3) Experimental group (Ad-S-F135N/N137T): mice were intramuscularly injected with a vaccine composition comprising 5×10⁷ pfu of adenovirus vector expressing F135N/N137T glycan-masking spike protein; (4) Experimental group (Ad-S-R158N/Y160T): mice were intramuscularly injected with a vaccine composition comprising 5×10⁷ pfu of adenovirus vector expressing R158N/Y160T glycan-masking spike protein; (5) Experimental group (Ad-S-N370/A372T): mice were intramuscularly injected with a vaccine composition comprising 5×10⁷ pfu of adenovirus vector expressing N370/A372T glycan-masking spike protein; (6) Experimental group (Ad-S-H519N/P521T): mice were intramuscularly injected with a vaccine composition comprising 5×10⁷ pfu of adenovirus vector expressing H519N/P521T glycan-masking spike protein; (7) Experimental group (Ad-S-N354/K356T): mice were intramuscularly injected with a vaccine composition comprising 1×10⁸ pfu of adenovirus vector expressing N354/K356T glycan-masking spike protein; (8) Experimental group (Ad-S-G413N): mice were intramuscularly injected with a vaccine composition comprising 1×10⁸ pfu of adenovirus vector expressing G413N glycan-masking spike protein; (9) Experimental group (Ad-S-D428N): mice were intramuscularly injected with a vaccine composition comprising 1×10⁸ pfu of adenovirus vector expressing D428N glycan-masking spike protein; (10) Experimental group (Ad-S-R21N/Q23T): mice were intramuscularly injected with a vaccine composition comprising 1×10⁸ pfu of adenovirus vector expressing R21N/Q23T glycan-masking spike protein; (11) Experimental group (Ad-S-P85N/N87T): mice were intramuscularly injected with a vaccine composition comprising 1×10⁸ pfu of adenovirus vector expressing P85N/N87T glycan-masking spike protein; (12) Experimental group (Ad-S-N87/G89T): mice were intramuscularly injected with a vaccine composition comprising 1×10⁸ pfu of adenovirus vector expressing N87/G89T glycan-masking spike protein; (13) Experimental group (Ad-S-H146N/N148T): mice were intramuscularly injected with a vaccine composition comprising 1×10⁸ pfu of adenovirus vector expressing H146N/N148T glycan-masking spike protein; (14) Experimental group (Ad-S-L179N/G181T): mice were intramuscularly injected with a vaccine composition comprising 1×10⁸ pfu of adenovirus vector expressing L179N/G181T glycan-masking spike protein; (15) Experimental group (Ad-S-Q183N/N185T): mice were intramuscularly injected with a vaccine composition comprising 1×10⁸ pfu of adenovirus vector expressing Q183N/N185T glycan-masking spike protein; (16) Experimental group (Ad-S-N185/K187T): mice were intramuscularly injected with a vaccine composition comprising 1×10⁸ pfu of adenovirus vector expressing N185/K187T glycan-masking spike protein; (17) Experimental group (Ad-S-V213N/D215T): mice were intramuscularly injected with a vaccine composition comprising 1×10⁸ pfu of adenovirus vector expressing V213N/D215T glycan-masking spike protein; (18) Experimental group (Ad-S-G219N): mice were intramuscularly injected with a vaccine composition comprising 1×10⁸ pfu of adenovirus vector expressing G219N glycan-masking spike protein; (19) Experimental group (Ad-S-D253N): mice were intramuscularly injected with a vaccine composition comprising 1×10⁸ pfu of adenovirus vector expressing D253N glycan-masking spike protein. Mice in each of the above groups were immunized for a total of two doses with an interval of 3 weeks between each dose.

2 weeks after the second dose of immune injection, the serum of mice in each group was harvested and collected to analyze the content of anti-spike protein IgG antibodies, anti-RBD IgG antibodies, and neutralizing antibodies against the original Wuhan strain of SARS-CoV-2.

Enzyme-linked immunosorbent assay (ELISA) was used to detect the titers of anti-spike protein IgG antibodies and anti-RBD IgG antibodies in serum samples. The detailed method is as follows. To measure the SARS-CoV-2 specific total IgG titer in the antisera, recombinant spike protein (Wuhan-Hu-1, catalog number 40589-V08H4) and recombinant RBD (Wuhan-Hu-1, cat number 40592-V08H) proteins were obtained from Sino Biological Inc., and allowed to coat 96-well plates at a concentration of 2 μg/well in coating buffer (100 μL/well) overnight at 4° C. Coating buffers were aspirated and washed three times with 300 μL of PBS containing 0.05% Tween 20 (PBST) to remove excess recombinant protein. Each well was blocked with 200 μL blocking buffer (1% BSA in PBS) at room temperature for 2 h to avoid non-specific binding. The plates were washed three times with 300 μL of PBST solution. Heat-inactivated serum samples were pre-diluted 1:1000, followed by 2-fold serial dilution in dilution buffer (0.05% tween 20 +1% BSA in PBST). Serially diluted serum samples were added to 96-well culture plates and incubated at room temperature for 1 hour to bind the antibody to spike protein or RBD immobilized on the 96-well culture plate. The plates were washed three times with 300 μL PBST. 100 μL of HRP conjugated anti-mouse IgG antibody (diluted with a dilution buffer solution at a ratio of 1:30000) was added to the 96-well culture plate, and incubated for 1 hour at room temperature in the dark. After three additional washes with 300 μL PBST, 100 μL of 3,3′,5,5′-Tetramethylbenzidine (TMB) substrate (BioLegend) of HRP was added to each well and incubated in the dark for 15 min. The reaction was stopped by the addition of 100 μL of 2 N H₂SO₄. The optical density at 450 nm was measured using a TECAN spectrophotometer. The end-point titration values were calculated in terms of a final serial dilution higher than 0.2 optical density value.

Pseudo-virus micro neutralization assay was used to detect neutralizing antibody titers against the original Wuhan strain of SARS-CoV-2 in serum samples. The detailed method is as follows. In each well of the 96-well culture plate, 10,000 HEK-293T cells stably expressing ACE2 were seeded in each well of the 96-well culture plate, followed by culturing in a cell culture incubator at 37° C. for one day. Serum samples from each group were serially diluted in DMEM containing 2% FBS. DMEM containing 1% FBS and 1% penicillin/streptomycin was used to react serially diluted serum samples with 1,000 TU (transducing units) pseudotyped lentivirus of the original Wuhan strain of SARS-CoV-2 at 37° C. for 1 hour. The action solution was added to the 96-well culture plate in the same volume to infect the aforementioned HEK-293T cells. The medium was changed to fresh complete DMEM (containing 10% FBS and 100 U/mL penicillin/streptomycin) 16 hours after infection, and the cells were cultured for an additional 48 hours. The cells were lysed and the ability of serum in each group to neutralize the virus (pseudo-neutralization) was calculated using Luciferase assay (Promega Bright-Glo™ Luciferase Assay System). The percentage of inhibition was calculated as the ratio of the loss of luciferase readout (RLU) in the presence of serum to that of the no serum control. The formula used for the calculation was (RLU Control−RLU Serum)/RLU Control. Neutralization titers (IC-50) were measured as the reciprocal of the serum dilution required to obtain a 50% reduction in RLU compared to a control containing the SARS-CoV-2 S-pseudotyped lentivirus only. Neutralization curves and IC-50 values were analyzed using the GraphPad Prism v6.01 Software.

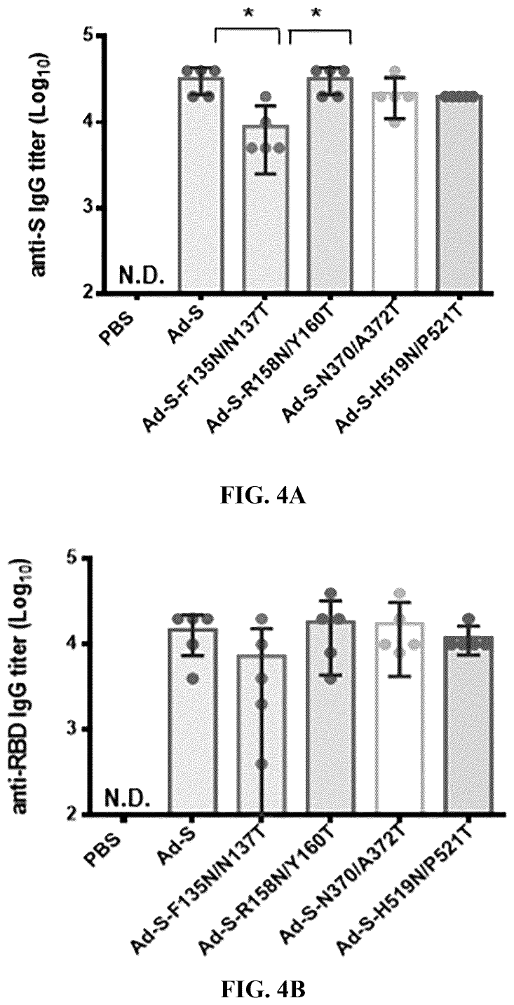

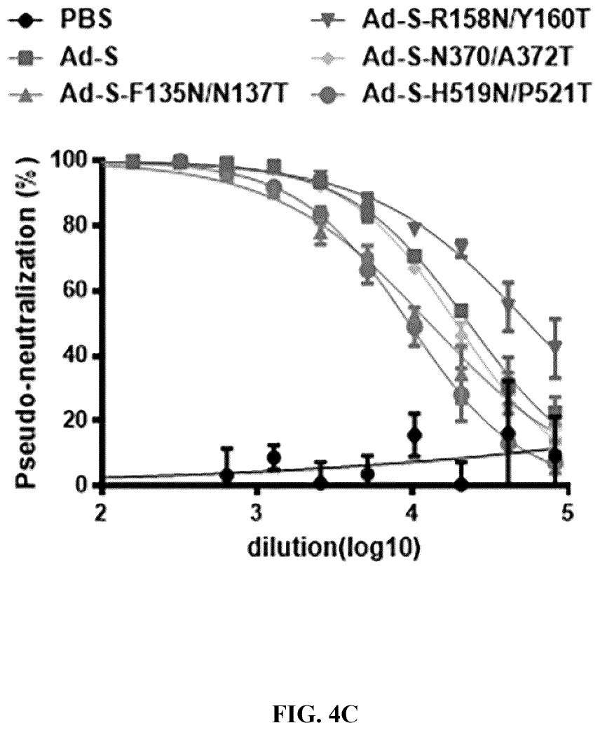

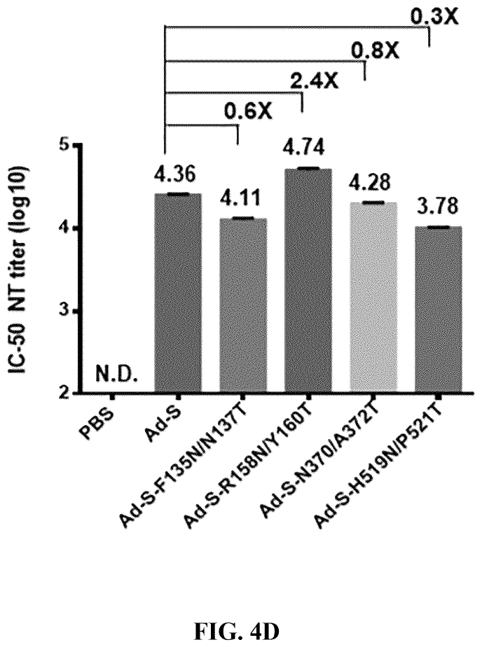

After immunization with the vaccine composition containing Ad-S-F135N/N137T, Ad-S-R158N/Y160T, Ad-S-N370/A372T and Ad-S-H519N/P521T, the titer of anti-spike protein IgG antibodies in mouse serum is as shown in FIG. 4A. * indicates p<0.05, N.D. indicates not detectable; The titers of anti-RBD IgG antibodies in mouse serum are shown in FIG. 4B, N.D. indicates not detectable; The ability of mouse sera to neutralize viral infection is shown in FIG. 4C, expressed as the percentage of inhibition of viral infection; The IC50 neutralizing titer of the antibody in mouse serum is shown in FIG. 4D, and the numerical multiple of the experimental group compared to the comparison group is expressed on a linear scale, N.D. indicates not detectable.

As can be seen from FIGS. 4A to 4D, after mice were immunized with Ad-S-F135N/N137T, the titers of anti-spike IgG antibodies against the original Wuhan strain of SARS-CoV-2 were significantly lower than those after immunization with Ad-S or Ad-S-R158N/Y160T. Compared to Ad-S, Ad-S-R158N/Y160T, Ad-S-N370/A372T or Ad-S-H519N/P521T, after mice were immunized with Ad-S-F135N/N137T, the titers of anti-RBD IgG antibodies against the original Wuhan strain of SARS-CoV-2 were also relatively low (not statistically significant). After immunization with Ad-S, Ad-S-F135N/N137T, Ad-S-R158N/Y160T, Ad-S-N370/A372T, or Ad-S-H519N/P521T, mouse serum has a dose-dependent ability to neutralize infection against the original Wuhan strain of SARS-CoV-2. The mice in the control group injected only with PBS solution do not have this phenomenon. Compared to immunization with Ad-S or Ad-S-N370/A372T, after mice are immunized with Ad-S-R158N/Y160T, their serum neutralizing antibody titers would increase. IC50 is approximately 2.4 times that of the comparison group. After immunization with Ad-S-F135N/N137T or Ad-S-H519N/P521T, the neutralizing antibody titer in the serum would decrease.

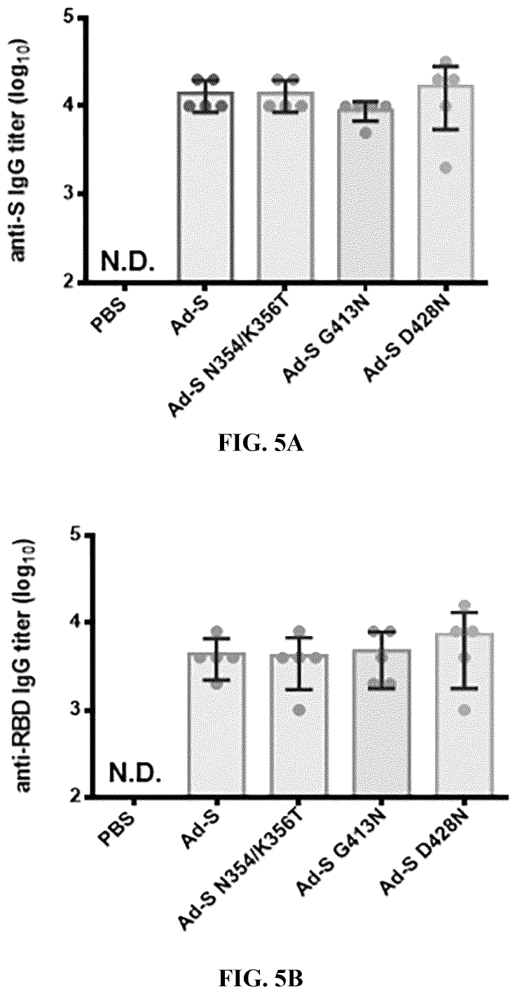

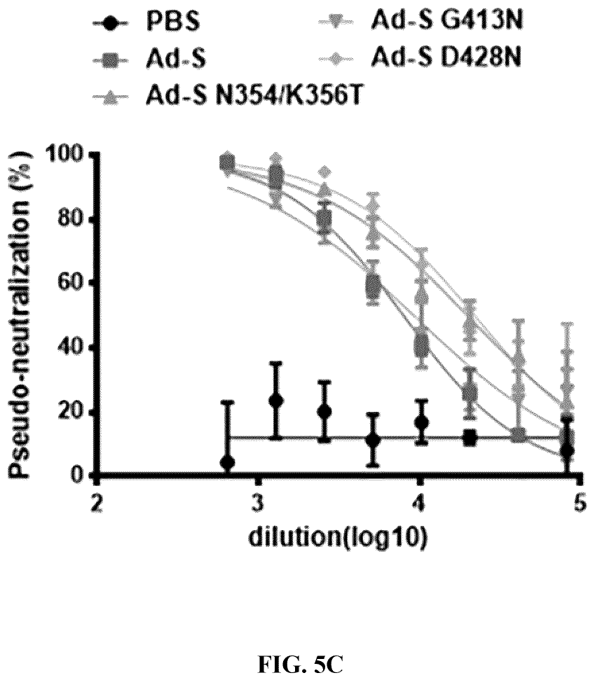

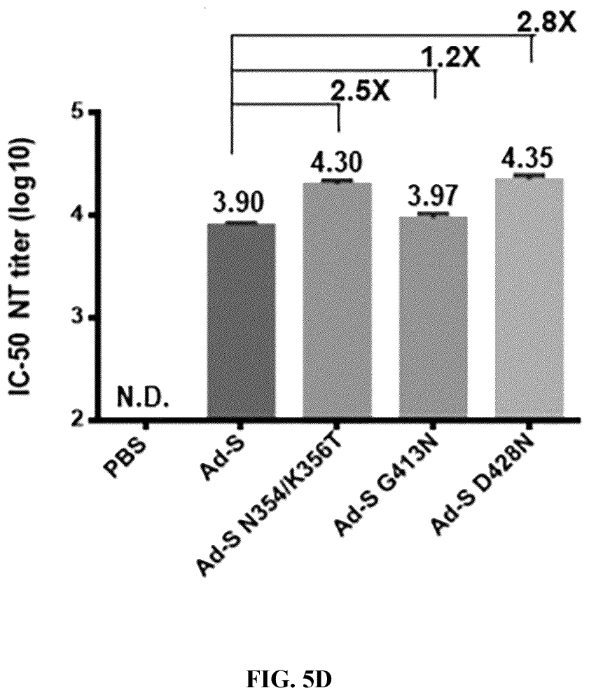

After immunization with the vaccine composition containing Ad-S-N354/K356T, Ad-S-G413N, and Ad-S-D428N, the titer of anti-spike protein IgG antibody in mouse serum is as shown in FIG. 5A. N.D. indicates not detectable. The titers of anti-RBD IgG antibodies in mouse serum are shown in FIG. 5B. N.D. indicates not detectable. The ability of mouse sera to neutralize viral infection is shown in FIG. 5C, expressed as the percentage of inhibition of viral infection; The IC50 neutralizing titer of the antibody in mouse serum is shown in FIG. 5D, and the numerical multiple of the experimental group compared to the comparison group is expressed on a linear scale, N.D. indicates not detectable.

As can be seen from FIGS. 5A to 5D, after mice were immunized with Ad-S-N354/K356T, Ad-S-N354/K356T, Ad-S-G413N or Ad-S-D428N, the titers of anti-spike IgG antibodies and anti-RBD IgG antibodies against the original Wuhan strain of SARS-CoV-2 were similar to those of mice immunized with Ad-S. After immunization with Ad-S, Ad-S-N370/K356T, Ad-S-G413N, or Ad-S-D428N, the ability of mouse serum to neutralize the original Wuhan strain of SARS-CoV-2 is dose-dependent. The mice in the control group injected only with PBS solution do not have this phenomenon. Compared to immunization with Ad-S, after mice are immunized with Ad-S-N354/K356T or Ad-S-D428N, the neutralizing antibody titers in their serum would increase. The IC50 was 2.5 times or 2.8 times that of the comparison group, respectively.

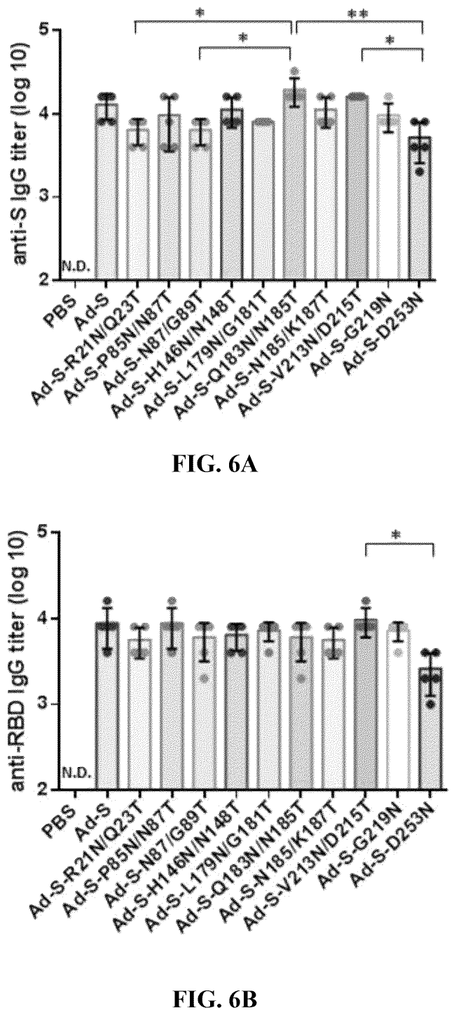

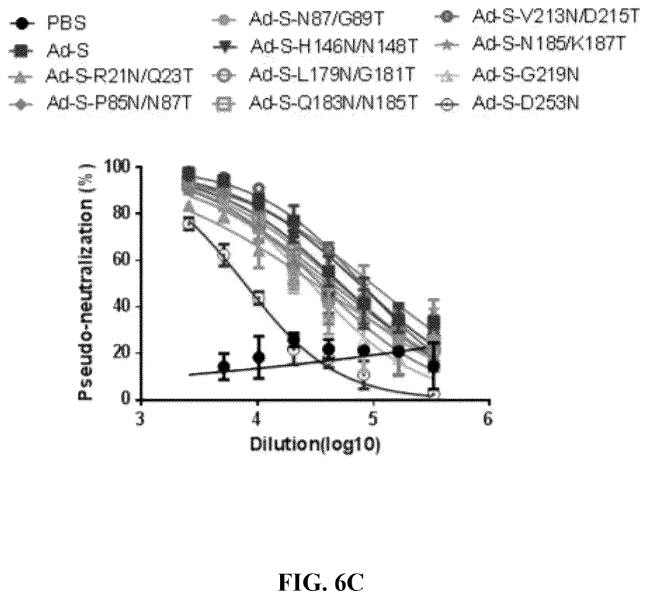

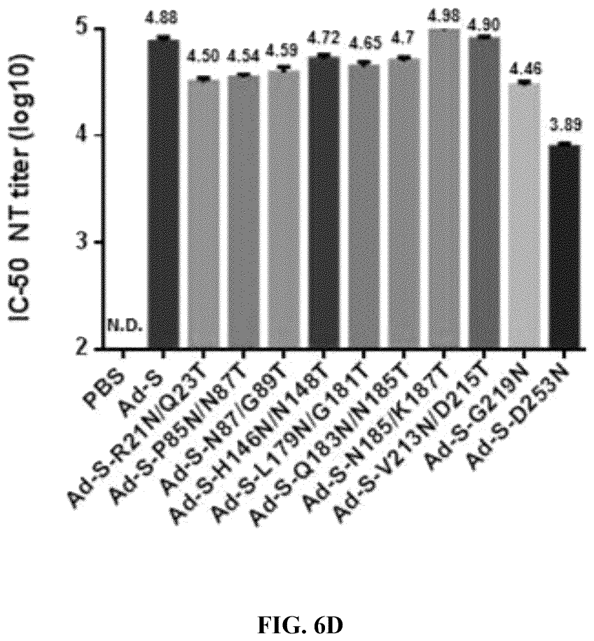

After immunization with the vaccine composition containing Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, and Ad-S-D253N, the titer of anti-spike protein IgG antibody in mouse serum is as shown in FIG. 6A. * indicates p<0.05, ** indicates p<0.01, N.D. indicates not detectable. The titers of anti-RBD IgG antibodies in mouse serum are shown in FIG. 6B. * indicates p<0.05, N.D. indicates not detectable. The ability of mouse sera to neutralize viral infection is shown in FIG. 6C, expressed as the percentage of inhibition of viral infection; The IC50 neutralizing titer of the antibody in mouse serum is shown in FIG. 6D, and the numerical multiple of the experimental group compared to the comparison group is expressed on a linear scale, N.D. indicates not detectable.

As can be seen from FIGS. 6A to 6D, after mice were immunized with Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, or Ad-S-D253N, the titers of anti-spike IgG antibodies and anti-RBD IgG antibodies against the original Wuhan strain of SARS-CoV-2 were similar to those of mice immunized with Ad-S (no statistically significant difference). After mice were immunized with Ad-S-Q183N/N185T, the titers of anti-spike IgG antibodies against the original Wuhan strain of SARS-CoV-2 were significantly higher than those in mice immunized with Ad-S-R21N/Q23T, Ad-S-N87/G89T or Ad-S-D253N. The titer of anti-spike IgG antibodies against the original Wuhan strain of SARS-CoV-2 elicited by mice immunized with Ad-S-V213N/D215T was significantly higher than that of mice immunized with Ad-S-D253N.

After immunization with Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, or Ad-S-D253N, the ability of mouse serum to neutralize the original Wuhan strain of SARS-CoV-2 is dose-dependent. The mice in the control group injected only with PBS solution do not have this phenomenon. Compared to immunization with Ad-S, after mice were immunized with Ad-S-N185/K187T or Ad-S-V213N/D215T, there was no significant difference in the neutralizing ability of their serum against the original Wuhan strain of SARS-CoV-2. After immunization with Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-G219N, or Ad-S-D253N, the neutralizing ability of the serum against the original Wuhan strain of SARS-CoV-2 would be reduced.

These results show that glycan-masking the R158N/Y160T site in the spike protein NTD of SARS-CoV-2, and glycan-masking the N354/K356T or D428N site in the spike protein RBD, can effectively refocus the antibody response of individual B cells to increase the titer of neutralizing antibodies produced. When used for immunization injections, they can effectively improve an individual's ability to resist infection by the original Wuhan strain of SARS-CoV-2.

### Example 3

### Mutant SARS-CoV-2 Spike Protein of Present Invention Improves Titer of Antibodies Against Variants

In one embodiment of the present invention, in order to further confirm that the mutant SARS-CoV-2 spike protein of the present invention can effectively induce mammals to produce antibody responses against different SARS-CoV-2 variants. Using the sera in each group of mice obtained in Example 2, ELISA was used to detect the titers of anti-spike protein IgG antibody and anti-RBD IgG antibody against the British variant (Alpha, B.1.1.7), the South African variant (Beta, B.1.351) and the Indian variant (Delta, B.1.617.2) of SARS-CoV-2 in the serum samples. Neutralizing antibody titers against the British variant (Alpha, B.1.1.7), the South African variant (Beta, B.1.351) and the Indian variant (Delta, B.1.617.2) of SARS-CoV-2 in serum samples were detected using a pseudo-virus micro neutralization assay.

### 3-1 British Variant (Alpha, B.1.1.7)

ELISA was used to detect anti-spike protein IgG antibody and anti-RBD IgG antibody titers in serum samples. The detailed method is as described in Example 2 and would not be described in detail here. However, the recombinant spike protein S1 subunit (Sino Biological Inc., catalog number 40591-VH12) and the recombinant RBD (Sino Biological Inc., catalog number 40592-V08H82) of the British variant (Alpha, B.1.1.7) of SARS-CoV-2 were coated on 96-well culture plate here.

The detailed method for detecting the neutralizing antibody titer of the British variant of SARS-CoV-2 in serum samples using the pseudo-virus micro neutralization assay is as described in Example 2 and would not be described in detail here. However, pseudotyped lentivirus expressing the full-length spike protein of the British variant of SARS-CoV-2 was used here.

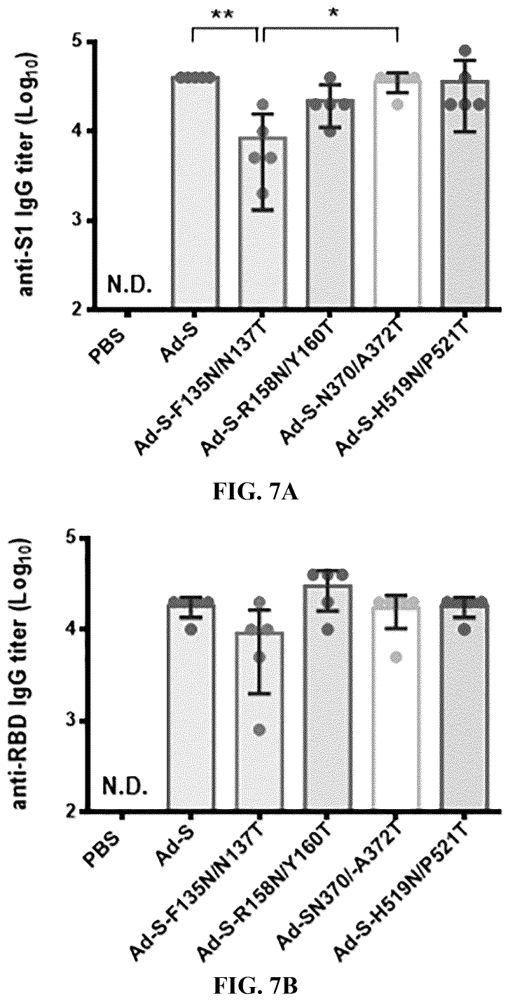

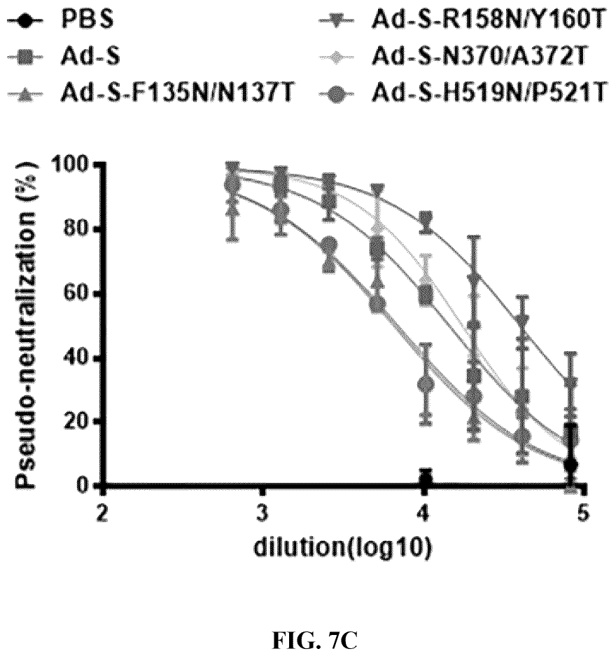

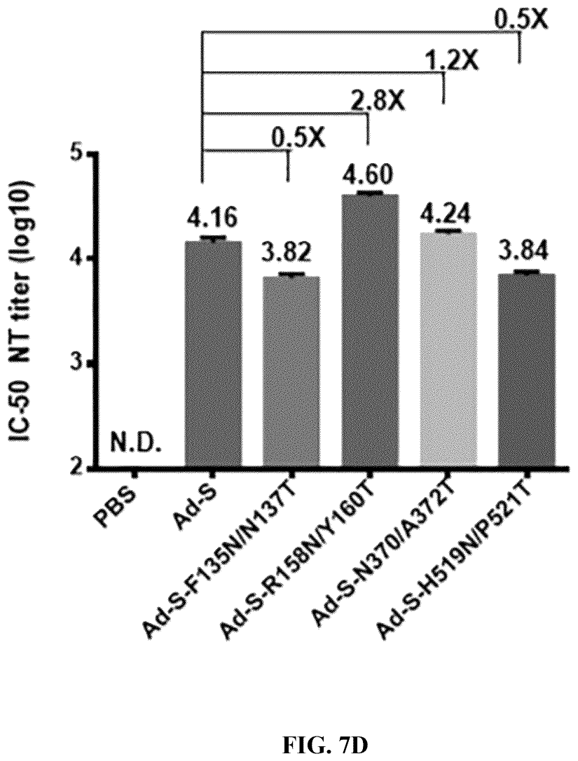

After immunization with the vaccine composition containing Ad-S-F135N/N137T, Ad-S-R158N/Y160T, Ad-S-N370/A372T, and Ad-S-H519N/P521T, the titer of anti-spike protein IgG antibodies in mouse serum is as shown in FIG. 7A. * indicates p<0.05, ** indicates p<0.01, N.D. indicates not detectable. * indicates p<0.05, ** indicates p<0.01, N.D. indicates not detectable. The titers of anti-RBD IgG antibodies in mouse serum are shown in FIG. 7B. N.D. indicates not detectable. The ability of mouse sera to neutralize viral infection is shown in FIG. 7C, expressed as the percentage of inhibition of viral infection; The IC50 neutralizing titer of the antibody in mouse serum is shown in FIG. 7D, and the numerical multiple of the experimental group compared to the comparison group is expressed on a linear scale, N.D. indicates not detectable.

As can be seen from FIGS. 7A to 7D, after mice were immunized with Ad-S-F135N/N137T, the titers of anti-spike IgG antibodies against the British variant of SARS-CoV-2 were significantly lower than those of mice immunized with Ad-S or Ad-S-N370/A372T. Compared to Ad-S, Ad-S-R158N/Y160T, Ad-S-N370/A372T, or Ad-S-H519N/P521T, after mice were immunized with Ad-S-F135N/N137T, the titers of anti-RBD IgG antibodies against the British variant of SARS-CoV-2 were also relatively low. Compared to immunization with Ad-S, Ad-S-F135N/N137T, Ad-S-N370/A372T, or Ad-S-H519N/P521T, after mice were immunized with Ad-S-R158N/Y160T, their serum had the best ability to neutralize infection against the British variant of SARS-CoV-2. The IC50 of serum neutralizing antibody titer was approximately 2.8 times that of the comparison group.

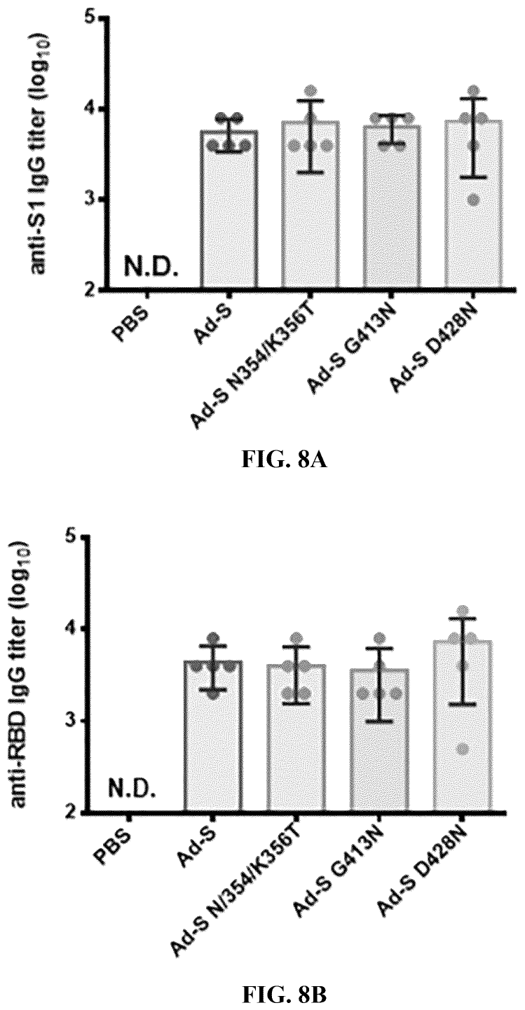

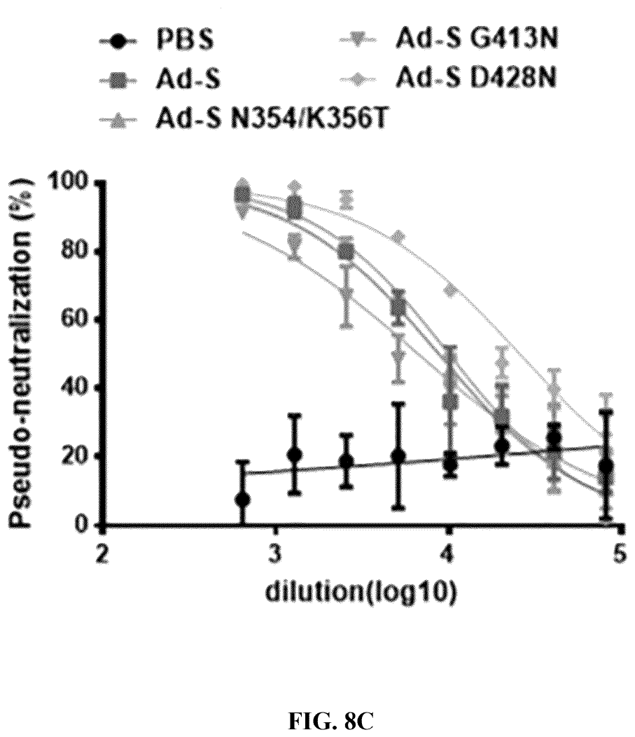

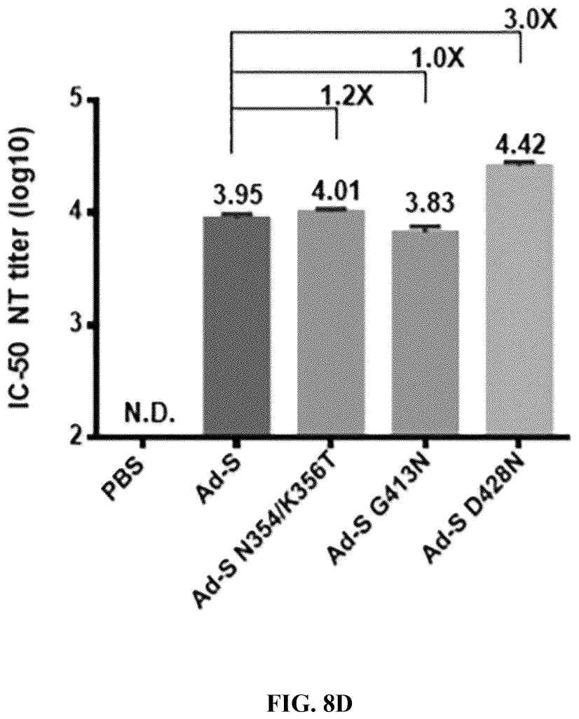

After immunization with the vaccine composition containing Ad-S-N354/K356T, Ad-S-G413N, and Ad-S-D428N, the titer of anti-spike protein IgG antibodies in mouse serum is as shown in FIG. 8A. N.D. indicates not detectable. The titers of anti-RBD IgG antibodies in mouse serum are shown in FIG. 8B. N.D. indicates not detectable. The ability of mouse sera to neutralize viral infection is shown in FIG. 8C, expressed as the percentage of inhibition of viral infection; The IC50 neutralizing titer of the antibody in mouse serum is shown in FIG. 8D, and the numerical multiple of the experimental group compared to the comparison group is expressed on a linear scale, N.D. indicates not detectable.

As can be seen from FIGS. 8A to 8D, after mice were immunized with Ad-S-N354/K356T, Ad-S-G413N, or Ad-S-D428N, the titers of anti-spike IgG antibodies and anti-RBD IgG antibodies against the British variant of SARS-CoV-2 were similar to those of mice immunized with Ad-S. Compared with immunization with Ad-S, Ad-S-N354/K356T, or Ad-S-G413N, after mice were immunized with Ad-S-D428N, the neutralizing ability of their serum against the British variant of SARS-CoV-2 can be effectively improved. The IC50 of serum neutralizing antibody titer was approximately 3.0 times that of the comparison group.

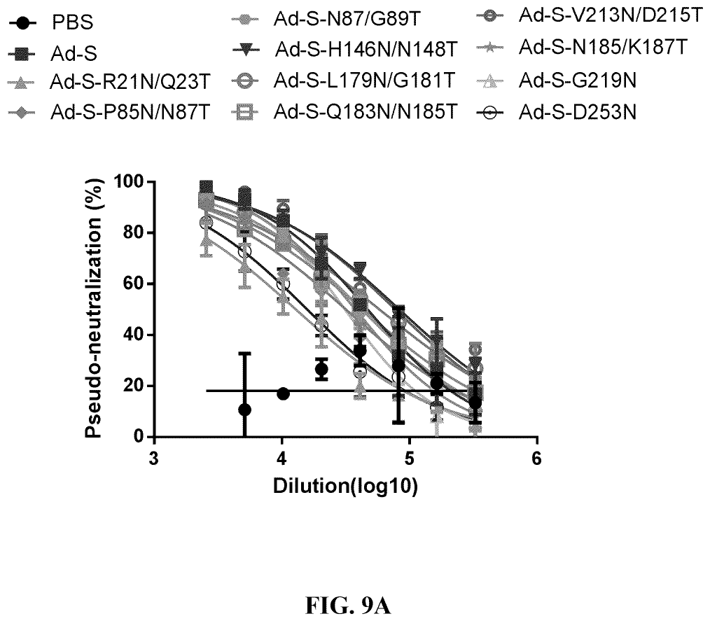

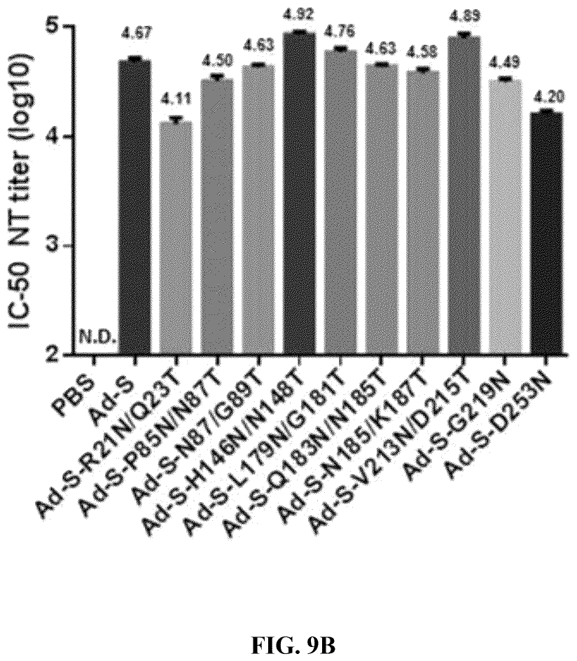

After immunization with the vaccine composition containing Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, and Ad-S-D253N, the ability of mouse sera to neutralize viral infection is shown in FIG. 9A, expressed as the percentage of inhibition of viral infection; The IC50 neutralizing titer of the antibody in mouse serum is shown in FIG. 9D, and the numerical multiple of the experimental group compared to the comparison group is expressed on a linear scale, N.D. indicates not detectable.

As can be seen from FIGS. 9A and 9B, compared to immunization with Ad-S, after mice were immunized with Ad-S-H146N/N148T or Ad-S-V213N/D215T, the neutralizing ability of their serum against the British variant of SARS-CoV-2 would be slightly improved. The IC50 of serum neutralizing antibody titer was 1.8 times and 1.7 times that of the comparison group respectively. After immunization with Ad-S-N87/G89T, Ad-S-L179N/G181T, Ad-S-Q183N/N185T or Ad-S-N185/K187T, there was no significant difference in the neutralizing antibody titer of the serum. After immunization with Ad-S-R21N/Q23T, Ad-S-P85N/N87T, Ad-S-G219N, or Ad-S-D253N, the neutralizing ability of the serum against the British variant of SARS-CoV-2 would be reduced.

These results show that glycan-masking the R158N/Y160T site in the spike protein NTD of SARS-CoV-2, and glycan-masking the D428N site in the spike protein RBD, can effectively refocus the antibody response of individual B cells to increase the neutralizing antibody titer against the British variant of SARS-CoV-2. When used for immunization injections, it can effectively improve an individual's ability to resist infection by the British variant of SARS-CoV-2.

### 3-2 South African Variant (Beta, B.1.351)

ELISA was used to detect anti-spike protein IgG antibody and anti-RBD IgG antibody titers in serum samples. The detailed method is as described in Example 2 and would not be described in detail here. However, the recombinant spike protein S1 subunit (Sino Biological Inc., catalog number 40591-V08H10) and the recombinant RBD (Sino Biological Inc., catalog number 40592-V08H85) of the South African variant (Beta, B.1.351) of SARS-CoV-2 were coated on 96-well culture plate here.

The detailed method for detecting the neutralizing antibody titer of the British variant of SARS-CoV-2 in serum samples using the pseudo-virus micro neutralization assay is as described in Example 2 and would not be described in detail here. However, pseudotyped lentivirus expressing the full-length spike protein of the South African variant of SARS-CoV-2 was used here.

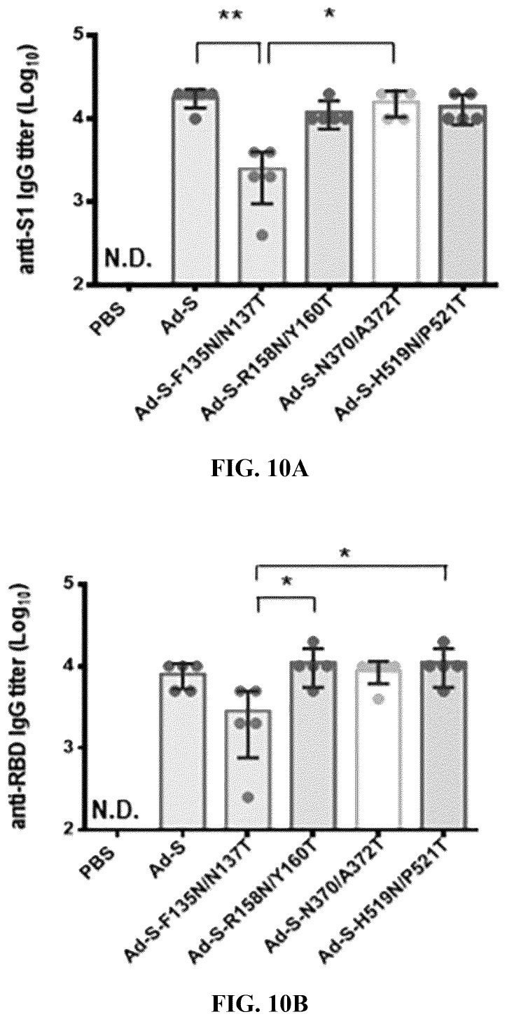

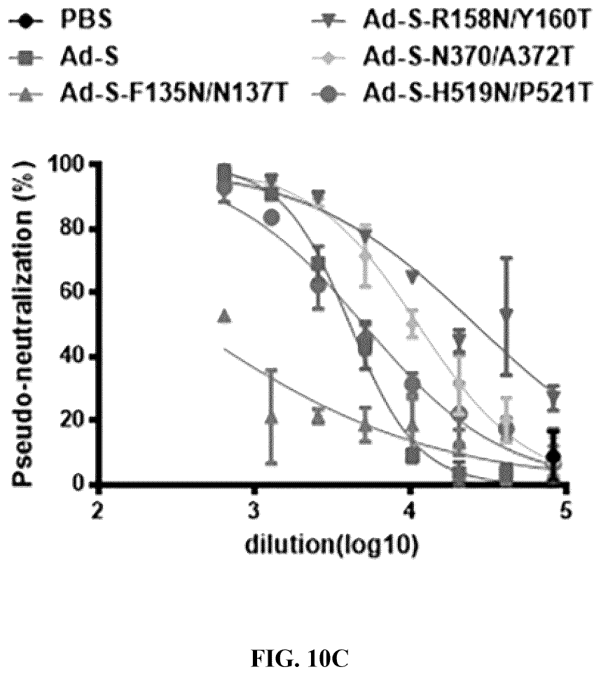

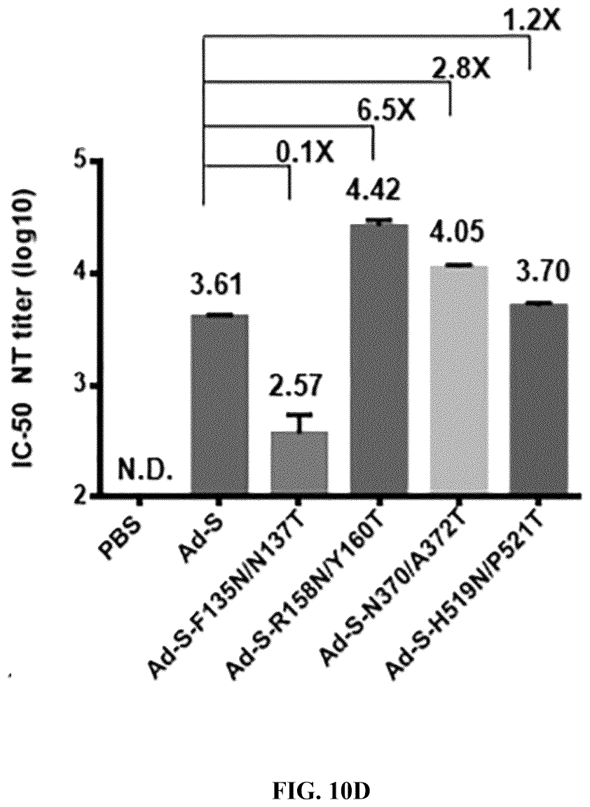

After immunization with the vaccine composition containing Ad-S-F135N/N137T, Ad-S-R158N/Y160T, Ad-S-N370/A372T, and Ad-S-H519N/P521T, the titer of anti-spike protein IgG antibodies in mouse serum is as shown in FIG. 10A. * indicates p<0.05, ** indicates p<0.01, N.D. indicates not detectable. The titers of anti-RBD IgG antibodies in mouse serum are shown in FIG. 10B. * indicates p<0.05, N.D. indicates not detectable. The ability of mouse sera to neutralize viral infection is shown in FIG. 10C, expressed as the percentage of inhibition of viral infection; The IC50 neutralizing titer of the antibody in mouse serum is shown in FIG. 10D, and the numerical multiple of the experimental group compared to the comparison group is expressed on a linear scale, N.D. indicates not detectable.

As can be seen from FIGS. 10A to 10D, after mice were immunized with Ad-S-F135N/N137T, the titers of anti-spike IgG antibodies against the South African variant of SARS-CoV-2 were significantly lower than those of mice immunized with Ad-S or Ad-S-N370/A372T. Compared to Ad-S-R158N/Y160T or Ad-S-H519N/P521T, after mice were immunized with Ad-S-F135N/N137T, the titers of anti-RBD IgG antibodies against the South African variant of SARS-CoV-2 were also relatively low. Compared to immunization with Ad-S, Ad-S-F135N/N137T or Ad-S-H519N/P521T, after mice were immunized with Ad-S-R158N/Y160T or Ad-S-N370/A372T, the ability to neutralize infection against the South African variant of SARS-CoV-2 in serum would be improved. The IC50 of serum neutralizing antibody titer was approximately 6.5 times or 2.8 times that of the comparison group, respectively.

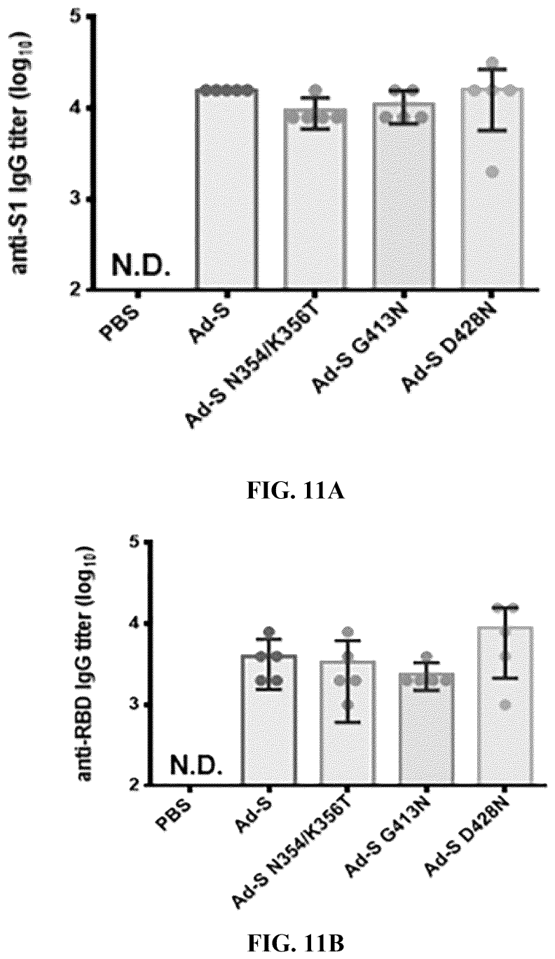

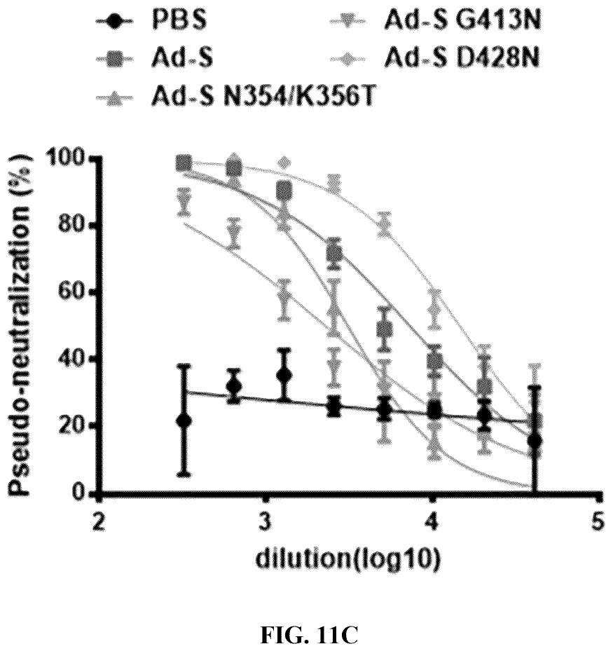

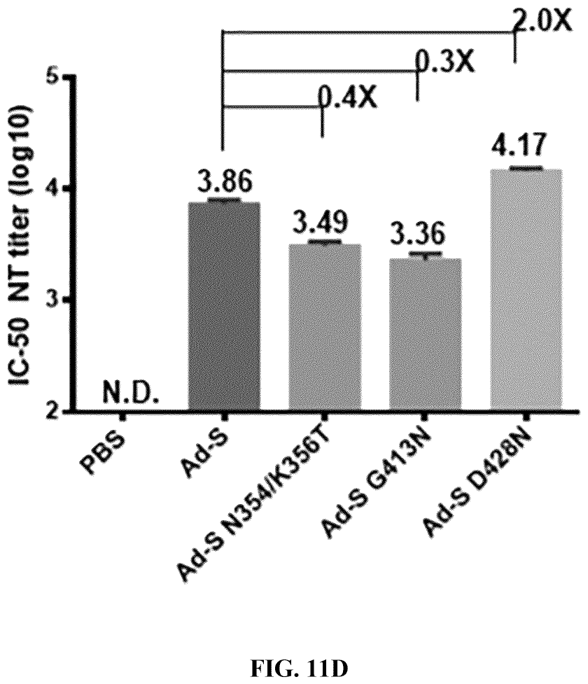

After immunization with the vaccine composition containing Ad-S-N354/K356T, Ad-S-G413N, and Ad-S-D428N, the titer of anti-spike protein IgG antibodies in mouse serum is as shown in FIG. 11A. N.D. indicates not detectable. The titers of anti-RBD IgG antibodies in mouse serum are shown in FIG. 11B. N.D. indicates not detectable. The ability of mouse sera to neutralize viral infection is shown in FIG. 11C, expressed as the percentage of inhibition of viral infection; The IC50 neutralizing titer of the antibody in mouse serum is shown in FIG. 11D, and the numerical multiple of the experimental group compared to the comparison group is expressed on a linear scale, N.D. indicates not detectable.

As can be seen from FIGS. 11A to 11D, after mice were immunized with Ad-S-N354/K356T, Ad-S-G413N, or Ad-S-D428N, the titers of anti-spike IgG antibodies and anti-RBD IgG antibodies against the South African variant of SARS-CoV-2 were similar to those of mice immunized with Ad-S. Compared with immunization with Ad-S, Ad-S-N354/K356T, or Ad-S-G413N, after mice were immunized with Ad-S-D428N, the neutralizing ability of their serum against the South African variant of SARS-CoV-2 can be effectively improved. The IC50 of serum neutralizing antibody titer was approximately 2.0 times that of the comparison group.

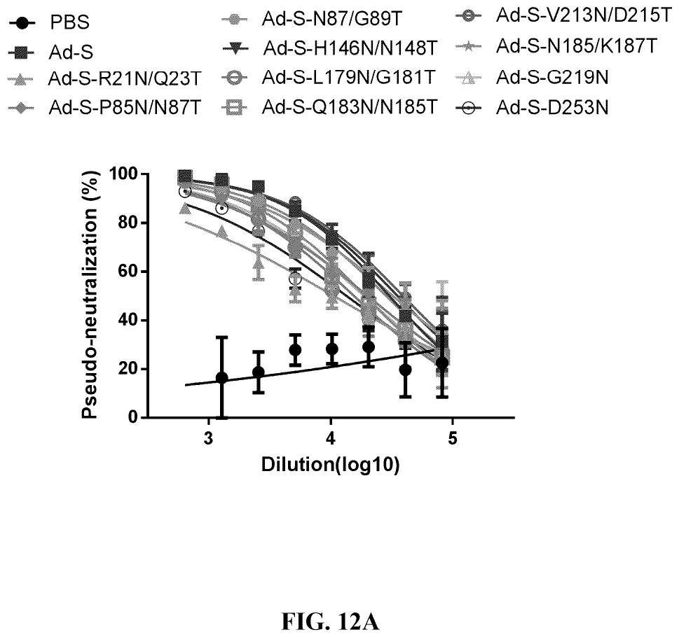

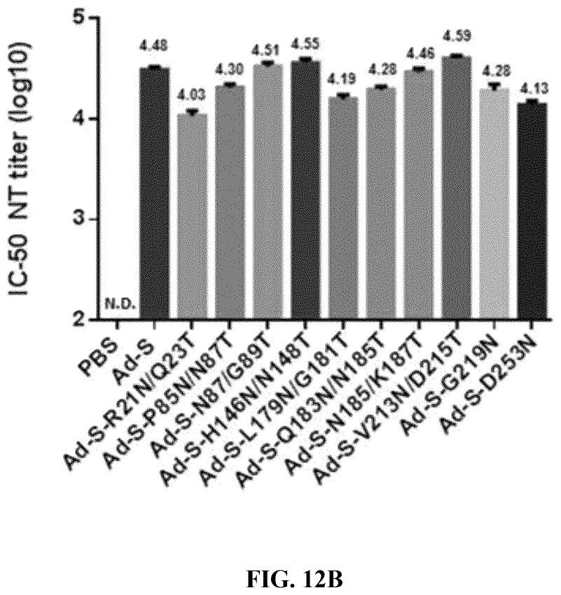

After immunization with the vaccine composition containing Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, and Ad-S-D253N, the ability of mouse sera to neutralize viral infection is shown in FIG. 12A, expressed as the percentage of inhibition of viral infection; The IC50 neutralizing titer of the antibody in mouse serum is shown in FIG. 12D, and the numerical multiple of the experimental group compared to the comparison group is expressed on a linear scale, N.D. indicates not detectable.

As can be seen from FIGS. 12A and 12B, compared to immunization with Ad-S, after mice were immunized with Ad-S-N87T/G89T, Ad-S-H146N/N148T, Ad-S-N185/K187T or Ad-S-V213N/D215T, the neutralizing ability of their serum against the South African variant of SARS-CoV-2 is no significant difference. After immunization with Ad-S-R21N/Q23T, Ad-S-P85N/N87T, Ad-S-L179N/G181T, Ad-S-Q183N/N185T, Ad-S-G219N, or Ad-S-D253N, the neutralizing ability of the serum against the South African variant of SARS-CoV-2 would be reduced.

These results show that glycan-masking the R158N/Y160T site in the spike protein NTD of SARS-CoV-2, and glycan-masking the N370/A372T site or the D428N site in the spike protein RBD, can effectively refocus the antibody response of individual B cells to increase the neutralizing antibody titer against the South African variant of SARS-CoV-2. When used for immunization injections, it can effectively improve an individual's ability to resist infection by the South African variant of SARS-CoV-2.

### 3-3 Indian Variant (Delta, B.1.617.2)

ELISA was used to detect anti-spike protein IgG antibody and anti-RBD IgG antibody titers in serum samples. The detailed method is as described in Example 2 and would not be described in detail here. However, the recombinant spike protein S1 subunit (Sino Biological Inc., catalog number 40591-V49H2-B) and the recombinant RBD (Sino Biological Inc., catalog number 40592-V08H90) of the South African variant (Beta, B.1.351) of SARS-CoV-2 were coated on 96-well culture plate here.

The detailed method for detecting the neutralizing antibody titer of the British variant of SARS-CoV-2 in serum samples using the pseudo-virus micro neutralization assay is as described in Example 2 and would not be described in detail here. However, pseudotyped lentivirus expressing the full-length spike protein of the Indian variant of SARS-CoV-2 was used here.

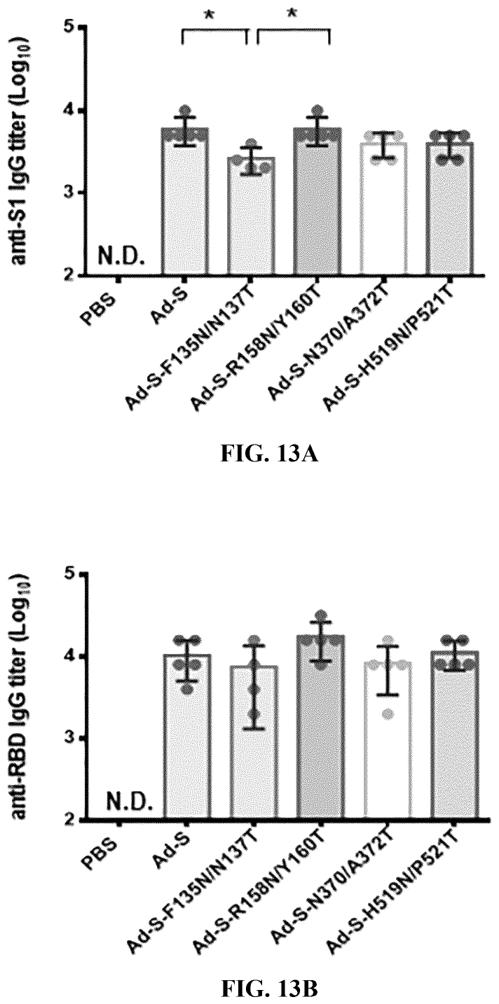

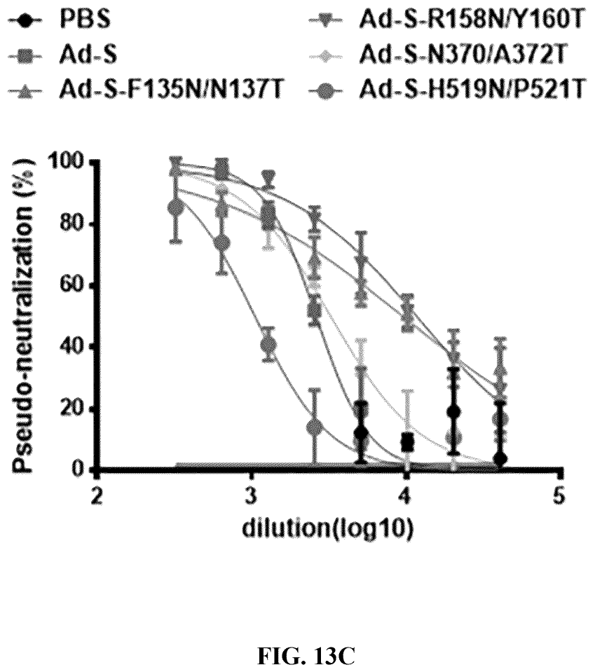

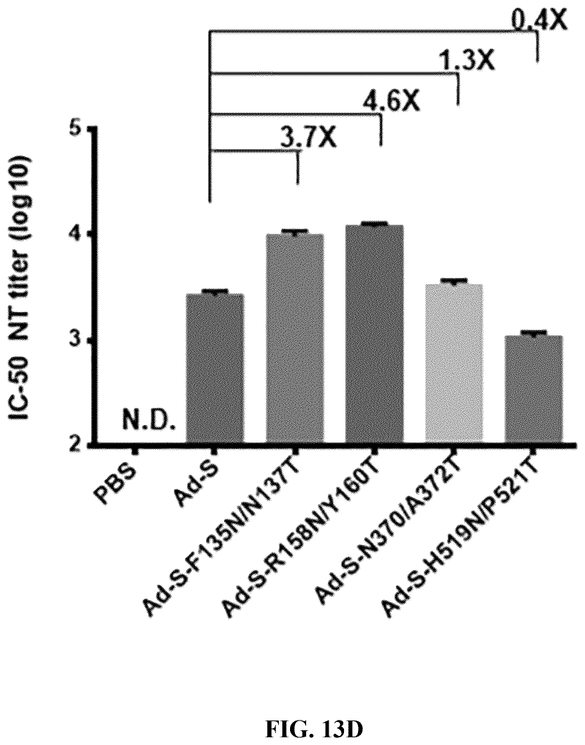

After immunization with the vaccine composition containing Ad-S-F135N/N137T, Ad-S-R158N/Y160T, Ad-S-N370/A372T, and Ad-S-H519N/P521T, the titer of anti-spike protein IgG antibodies in mouse serum is as shown in FIG. 13A. * indicates p<0.05, N.D. indicates not detectable. The titers of anti-RBD IgG antibodies in mouse serum are shown in FIG. 13B. N.D. indicates not detectable. The ability of mouse sera to neutralize viral infection is shown in FIG. 13C, expressed as the percentage of inhibition of viral infection; The IC50 neutralizing titer of the antibody in mouse serum is shown in FIG. 13D, and the numerical multiple of the experimental group compared to the comparison group is expressed on a linear scale, N.D. indicates not detectable.

As can be seen from FIGS. 13A to 13D, after mice were immunized with Ad-S-F135N/N137T, the titers of anti-spike IgG antibodies against the Indian variant of SARS-CoV-2 were significantly lower than those of mice immunized with Ad-S or Ad-S-R158N/Y160T. After mice were immunized with Ad-S-F135N/N137T, Ad-S-R158N/Y160T, Ad-S-N370/A372T, or Ad-S-H519N/P521T, the titers of anti-RBD IgG antibodies against the Indian variant of SARS-CoV-2 were similar to those of mice immunized with Ad-S. Compared to immunization with Ad-S, Ad-S-N370/A372T or Ad-S-H519N/P521T, after mice were immunized with Ad-S-F135N/N137T or Ad-S-R158N/Y160T, the ability to neutralize infection against the Indian variant of SARS-CoV-2 in serum would be improved. The IC50 of serum neutralizing antibody titer was approximately 3.7 times or 4.6 times that of the comparison group, respectively.

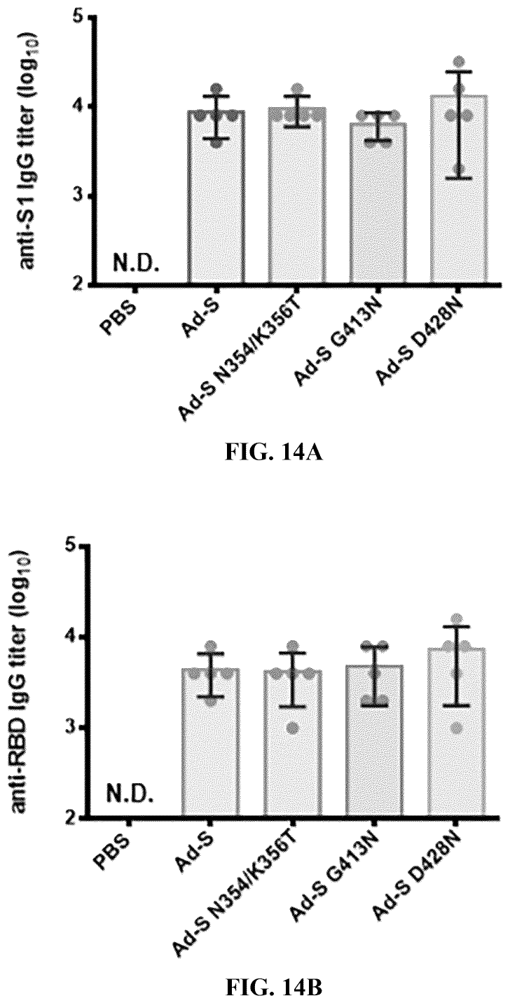

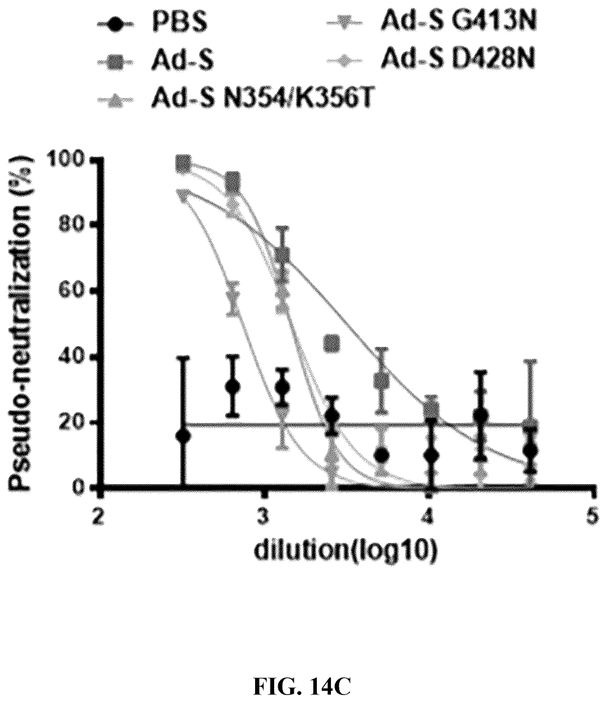

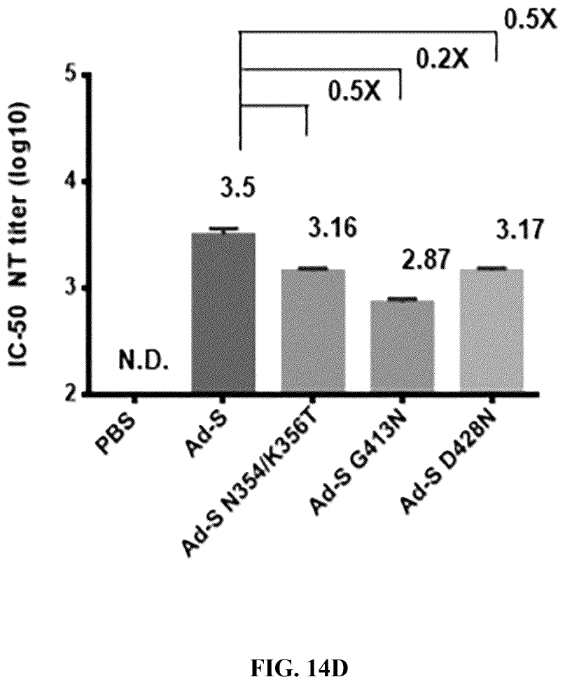

After immunization with the vaccine composition containing Ad-S-N354/K356T, Ad-S-G413N, and Ad-S-D428N, the titer of anti-spike protein IgG antibodies in mouse serum is as shown in FIG. 14A. N.D. indicates not detectable. The titers of anti-RBD IgG antibodies in mouse serum are shown in FIG. 14B. N.D. indicates not detectable. The ability of mouse sera to neutralize viral infection is shown in FIG. 14C, expressed as the percentage of inhibition of viral infection; The IC50 neutralizing titer of the antibody in mouse serum is shown in FIG. 14D, and the numerical multiple of the experimental group compared to the comparison group is expressed on a linear scale, N.D. indicates not detectable.

As can be seen from FIGS. 14A to 14D, after mice were immunized with Ad-S-N354/K356T, Ad-S-G413N, or Ad-S-D428N, the titers of anti-spike IgG antibodies, anti-RBD IgG antibodies, and the neutralizing ability against the Indian variant of SARS-CoV-2 were lower than those of mice immunized with Ad-S.

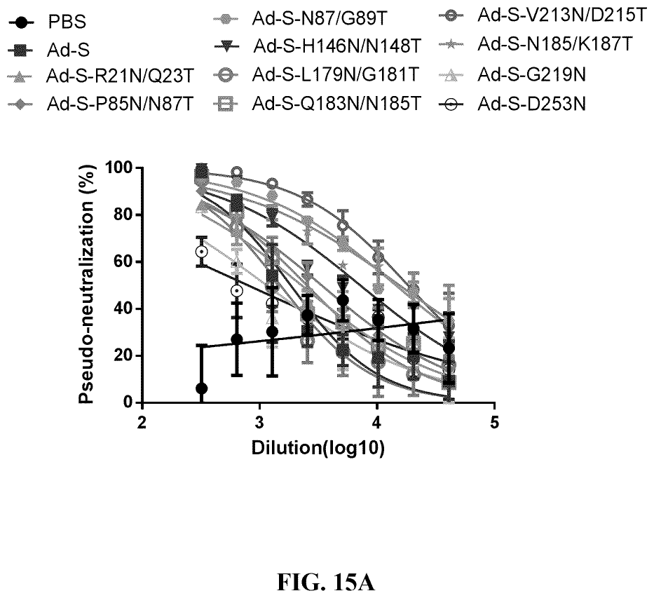

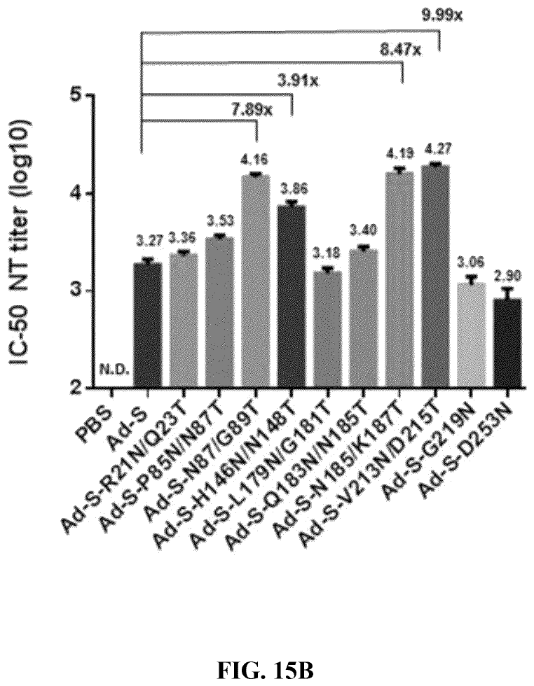

After immunization with the vaccine composition containing Ad-S-R21N/Q23T, Ad-S-P85N/N87T, Ad-S-N87/G89T, Ad-S-H146N/N148T, Ad-S-L179N/G181T, Ad-S-Q183N/N185T, Ad-S-N185/K187T, Ad-S-V213N/D215T, Ad-S-G219N, and Ad-S-D253N, the ability of mouse sera to neutralize viral infection is shown in FIG. 15A, expressed as the percentage of inhibition of viral infection; The IC50 neutralizing titer of the antibody in mouse serum is shown in FIG. 15D, and the numerical multiple of the experimental group compared to the comparison group is expressed on a linear scale, N.D. indicates not detectable.

As can be seen from FIGS. 15A and 15B, compared to immunization with Ad-S, after mice were immunized with Ad-S-N87T/G89T, Ad-S-H146N/N148T, Ad-S-N185/K187T, or Ad-S-V213N/D215T, the neutralizing ability of their serum against the Indian variant of SARS-CoV-2 would be significantly improved. The IC50 of serum neutralizing antibody titer was 7.9 times, 3.9 times, 8.5 times or 10.0 times that of the comparison group respectively. After immunization with Ad-S-P85N/N87T or Ad-S-Q183N/N185T, the ability to neutralize infection against the Indian variant of SARS-CoV-2 in serum would be slightly improved. The IC50 of the serum neutralizing antibody titer was 1.8 times or 1.4 times that of the comparison group respectively. After immunization with Ad-S-R21N/Q23T, Ad-S-L179N/G181T, Ad-S-Q183N/N185T, Ad-S-G219N, or Ad-S-D253N, the neutralizing ability of the serum against the Indian variant of SARS-CoV-2 would be no significant difference. After immunization with Ad-S-G219N or Ad-S-D253N, the neutralizing ability of the serum against the Indian variant of SARS-CoV-2 would be reduced.

These results show that glycan-masking the F135N/N137T site, the R158N/Y160T site, the N87T/G89T site, the H146N/N148T site, the N185/K187T site, or the V213N/D215T site in the spike protein NTD of SARS-CoV-2, can effectively refocus the antibody response of individual B cells to increase the neutralizing antibody titer against the Indian variant of SARS-CoV-2. When used for immunization injections, it can effectively improve an individual's ability to resist infection by the Indian variant of SARS-CoV-2.

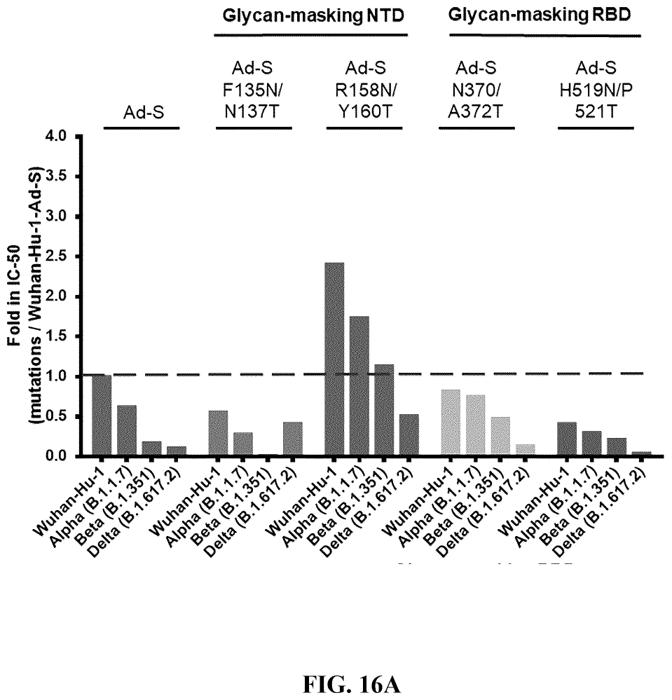

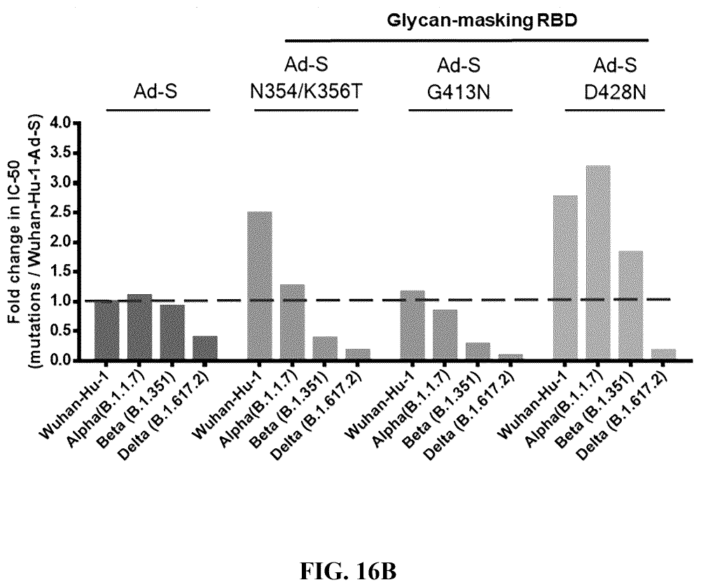

In the embodiment of the present invention, in order to directly compare the neutralizing antibody titers of different SARS-CoV-2 variants caused by mutant SARS-CoV-2 spike proteins, the IC50 neutralizing titers of antibodies in the serum of mice immunized with Ad-S-F135N/N137T, Ad-S-R158N/Y160T, Ad-S-N370/A372T, and Ad-S-H519N/P521T are also presented in FIG. 16A. The IC50 neutralizing titers of antibodies in the serum of mice immunized with Ad-S-N354/K356T, Ad-S-G413N, and Ad-S-D428N are also presented in FIG. 16B. Data were normalized using IC50 neutralizing titers of antibodies in sera of mice immunized with Ad-S.

As can be seen from FIG. 16A, the glycan-masking spike protein at the R158N/Y160T site has the best effect. Compared with wild-type spike protein, the IC50 titer of neutralizing antibodies against the original Wuhan strain was increased by 2.5 times. The IC50 titer of neutralizing antibodies against the British variant was increased by 1.8 times. The IC50 titer of neutralizing antibodies against the South African variant was increased by 1.2 times. Although the IC50 titer of neutralizing antibodies against the Indian variant was reduced to 0.6 times, it was still significantly higher than the neutralizing antibody titer of wild-type spike protein against the Indian variant. The result shows that glycan-masking the R158N/Y160T site in the spike protein NTD of SARS-CoV-2 can more effectively increase the titer of neutralizing antibodies against the original Wuhan strain, and can increase the potency of cross-neutralizing antibodies against the British variant, the South African variant, and the Indian variant.

As can be seen from FIG. 16B, the glycan-masking spike protein at the D428N site has the best effect. Compared with wild-type spike protein, the IC50 titer of neutralizing antibodies against the original Wuhan strain was increased by 2.7 times. The IC50 titer of neutralizing antibodies against the British variant was increased by 3.2 times. The IC50 titer of neutralizing antibodies against the South African variant was increased by 2.0 times. However, the IC50 titer of neutralizing antibodies against the Indian variant was reduced to 0.2 times. The result shows that glycan-masking the D428N site in the spike protein RBD of SARS-CoV-2 can more effectively increase the titer of neutralizing antibodies against the original Wuhan strain, and can increase the potency of cross-neutralizing antibodies against the British variant and the South African variant.

In summary, in the SARS-CoV-2 vaccine composition of the present invention, a mutant SARS-CoV-2 spike protein that is overly glycosylated in the NTD or RBD is used to glycan mask unimportant epitopes, so that the response of individual B cells to the antibody of the SARS-CoV-2 spike protein can refocused without affecting the overall folding structure of the spike protein. The mutant SARS-CoV-2 spike protein of the present invention can effectively induce neutralizing antibody titers in individuals against the original Wuhan strain, the British variant, the South African variant, and the Indian variant of the SARS-CoV-2, to effectively improve an individual's ability to resist infection by different variants of SARS-CoV-2.

What is claimed is:

1. A mutant SARS-CoV-2 spike protein, comprising an N-linked glycosylation masking N-terminal domain and/or receptor-binding domain of SARS-CoV-2 spike protein.

2. The mutant SARS-CoV-2 spike protein according to claim 1, having a mutation at an amino acid residue of a wild-type SARS-CoV-2 spike protein, wherein the amino acid residue is selected from the group consisting of: amino acid residue 21, amino acid residue 23, amino acid residue 85, amino acid residue 87, amino acid residue 89, amino acid residue 135, amino acid residue 137, amino acid residue 146, amino acid residue 148, amino acid residue 158, amino acid residue 160, amino acid residue 179, amino acid residue 181, amino acid residue 183, amino acid residue 185, amino acid residue 187, amino acid residue 213, amino acid residue 215, amino acid residue 219, amino acid residue 253, amino acid residue 354, amino acid residue 356, amino acid residue 370, amino acid residue 413, amino acid residue 428, amino acid residue 519, and amino acid residue 521.

3. The mutant SARS-CoV-2 spike protein according to claim 2, wherein the mutation is that substituting the amino acid residue with asparagine or threonine.

4. The mutant SARS-CoV-2 spike protein according to claim 3, wherein the amino acid residue 21 and the amino acid residue 23 of the wild-type SARS-CoV-2 spike protein have asparagine and threonine substitutions respectively, the amino acid residue 85 and the amino acid residue 87 have asparagine and threonine substitutions respectively, the amino acid residue 89 has threonine substitution, the amino acid residue 135 and the amino acid residue 137 have asparagine and threonine substitutions respectively, the amino acid residue 146 and the amino acid residue 148 have asparagine and threonine substitutions respectively, the amino acid residue 158 and the amino acid residue 160 have asparagine and threonine substitutions respectively, the amino acid residue 179 and the amino acid residue 181 have asparagine and threonine substitutions respectively, the amino acid residue 183 and the amino acid residue 185 have asparagine and threonine substitutions respectively, the amino acid residue 187 has threonine substitution, the amino acid residue 213 and the amino acid residue 215 have asparagine and threonine substitutions respectively, the amino acid residue 219 has asparagine substitution, the amino acid residue 253 has asparagine substitution, the amino acid residue 356 has threonine substitution, the amino acid residue 372 has threonine substitution, the amino acid residue 413 has asparagine substitution, the amino acid residue 428 has asparagine substitution, or the amino acid residue 519 and the amino acid residue 521 have asparagine and threonine substitutions respectively.

5. A nucleic acid molecule, comprising a nucleotide sequence encoding the mutant SARS-CoV-2 spike protein according to claim 4.

6. A vaccine composition, comprising the mutant SARS-CoV-2 spike protein according tom claim 1.

7. The vaccine composition according to claim 6, wherein the mutant SARS-CoV-2 spike protein is expressed on a recombinant virus.

8. The vaccine composition according to claim 7, wherein the recombinant virus comprises the nucleic acid molecule according to claim 5.

9. The vaccine composition according to claim 7, wherein the recombinant virus is a recombinant adenovirus.

10. A method for preparing a SARS-CoV-2 vaccine composition, comprising using the mutant SARS-CoV-2 spike protein according to claim 1.

11. The method according to claim 10, wherein the SARS-CoV-2 vaccine composition elicits an immune response against multiple SARS-CoV-2 variants in an individual.

12. The method according to claim 10, wherein the SARS-CoV-2 vaccine composition elicits high-titer antigen-specific antibodies and/or neutralizing antibodies.

13. The vaccine composition according to claim 6, wherein the mutant SARS-CoV-2 spike protein has a mutation at an amino acid residue of a wild-type SARS-CoV-2 spike protein, wherein the amino acid residue is selected from the group consisting of: amino acid residue 21, amino acid residue 23, amino acid residue 85, amino acid residue 87, amino acid residue 89, amino acid residue 135, amino acid residue 137, amino acid residue 146, amino acid residue 148, amino acid residue 158, amino acid residue 160, amino acid residue 179, amino acid residue 181, amino acid residue 183, amino acid residue 185, amino acid residue 187, amino acid residue 213, amino acid residue 215, amino acid residue 219, amino acid residue 253, amino acid residue 354, amino acid residue 356, amino acid residue 370, amino acid residue 413, amino acid residue 428, amino acid residue 519, and amino acid residue 521.

14. The vaccine composition according to claim 13, wherein the mutation is that substituting the amino acid residue with asparagine or threonine.

15. The vaccine composition according to claim 14, wherein the amino acid residue 21 and the amino acid residue 23 of the wild-type SARS-CoV-2 spike protein have asparagine and threonine substitutions respectively, the amino acid residue 85 and the amino acid residue 87 have asparagine and threonine substitutions respectively, the amino acid residue 89 has threonine substitution, the amino acid residue 135 and the amino acid residue 137 have asparagine and threonine substitutions respectively, the amino acid residue 146 and the amino acid residue 148 have asparagine and threonine substitutions respectively, the amino acid residue 158 and the amino acid residue 160 have asparagine and threonine substitutions respectively, the amino acid residue 179 and the amino acid residue 181 have asparagine and threonine substitutions respectively, the amino acid residue 183 and the amino acid residue 185 have asparagine and threonine substitutions respectively, the amino acid residue 187 has threonine substitution, the amino acid residue 213 and the amino acid residue 215 have asparagine and threonine substitutions respectively, the amino acid residue 219 has asparagine substitution, the amino acid residue 253 has asparagine substitution, the amino acid residue 356 has threonine substitution, the amino acid residue 372 has threonine substitution, the amino acid residue 413 has asparagine substitution, the amino acid residue 428 has asparagine substitution, or the amino acid residue 519 and the amino acid residue 521 have asparagine and threonine substitutions respectively.

16. A method for improving ability to resist SARS-CoV-2 infection, comprising administering to a subject in need thereof a medicament comprising an effective amount of the vaccine composition according to claim 6.
